(19) United States
(12) Patent Application Publication
(10) Pub. No.: US 2026/0130629 A1
(43) Pub. Date: May 14, 2026

(54) STEERING WHEEL DETECTION DEVICE

(71) Applicant: Panasonic Automotive Systems Co., Ltd., Kanagawa (JP)

(72) Inventors: Yasuhiro YAMASHITA, Osaka (JP); Motoyuki OKAYAMA, Osaka (JP); Hideaki ETO, Osaka (JP)

(73) Assignee: Panasonic Automotive Systems Co., Ltd., Kanagawa (JP)

(21) Appl. No.: 19/357,920

(22) Filed: Oct. 14, 2025

(30) Foreign Application Priority Data
Nov. 14, 2024 (JP) 2024-198781

(51) Int. Cl. A61B 5/00; A61B 5/0205; A61B 5/0245; A61B 5/18; B62D 1/04; G01D 5/24

(52) U.S. Cl. CPC A61B 5/6893; A61B 5/0205; A61B 5/18; B62D 1/046; G01D 5/24; A61B 5/0245; A61B 5/4266; A61B 2560/0468; A61B 2562/04

(57) ABSTRACT

A steering wheel detection device includes: a first heart rate detection electrode, a first sweat detection electrode, a first heart rate and sweat detection electrode, and a reference electrode that are disposed in a rim. The first heart rate detection electrode is disposed on one of a right side or a left side of the rim. The first heart rate and sweat detection electrode is disposed on an other of the left side or the right side of the rim. The first sweat detection electrode is disposed adjacent to and not in contact with the first heart rate and sweat detection electrode. The reference electrode is disposed on at least one of the right side or the left side of the rim, the reference electrode not being in contact with the first heart rate detection electrode, the first sweat detection electrode, or the first heart rate and sweat detection electrode.

### CROSS REFERENCE TO RELATED APPLICATION

The present application is based on and claims priority of Japanese Patent Application No. 2024-198781 filed on Nov. 14, 2024.

### FIELD

The present disclosure relates to steering wheel detection devices.

### BACKGROUND

A heart rate signal measurement device that measures heart rate signals of a driver in operation using electrodes provided in the steering wheel of the vehicle is known as a conventional technique.

For example, Patent Literature (PTL) 1 discloses a driver's heart rate measurement device including: a positive electrode, a negative electrode, and a reference electrode for reducing a baseline drift that are disposed on a grip of a steering wheel to obtain heart rate signals from the hands of a driver; and a detection circuit connected to each of those electrodes. In the driver's heart rate signal measurement device, a heater disposed on the grip of the steering wheel is caused to produce heat to stimulate sweating of the palms of the hands gripping the steering wheel and stabilize a contact resistance between each electrode and the palms of the hands in a low state, thereby improving the reliability of results of measuring heart rate signals.

### CITATION LIST

### Patent Literature

PTL 1: Japanese Unexamined Patent Application Publication No. 2000-23929

### SUMMARY

However, the driver's heart rate signal measurement device according to PTL 1 can be improved upon.

In view of this, the present disclosure provides a steering wheel detection device capable of improving upon the above related art.

A steering wheel detection device according to one aspect of the present disclosure includes a first heart rate detection electrode, a first sweat detection electrode, a first heart rate and sweat detection electrode, and a reference electrode that are disposed in a rim of a steering wheel provided in a vehicle. In a plan view of the rim in a neutral position: the first heart rate detection electrode is disposed on one of a right side or a left side of the rim; the first heart rate and sweat detection electrode is disposed on an other of the left side or the right side of the rim; and the first sweat detection electrode is disposed adjacent to and not in contact with the first heart rate and sweat detection electrode. In the plan view of the rim in the neutral position, the reference electrode is disposed on at least one of the right side or the left side of the rim, the reference electrode not being in contact with the first heart rate detection electrode, the first sweat detection electrode, or the first heart rate and sweat detection electrode.

The steering wheel detection device of the present disclosure is capable of improving upon the above related art.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1A is a schematic diagram showing an example of a vehicle in which a steering wheel according to an embodiment is provided.

FIG. 1B Is a front view of a different steering wheel.

FIG. 2 is a block diagram showing a steering wheel detection device according to the embodiment.

FIG. 3 is a diagram showing: a rim in which each of electrodes that are a first heart rate detection electrode, a first sweat detection electrode, a first heart rate and sweat detection electrode, and a reference electrode shown in FIG. 2 is disposed; and cross sections of the rim.

FIG. 4 is a block diagram showing a steering wheel detection device in which a first sweat detection electrode, a first heart rate and sweat detection electrode, and a reference electrode are disposed on one of sides of a rim and a first heart rate detection electrode is disposed on the other of the sides of the rim.

FIG. 5 is a diagram showing: the rim in which each of the electrodes shown in FIG. 4 is disposed; and cross sections of the rim.

FIG. 6 is a block diagram showing a steering wheel detection device in which second heart rate detection electrodes are disposed in respective spokes.

FIG. 7 is a diagram showing: a rim in which each of the electrodes shown in FIG. 6 is disposed; and cross sections of the rim.

FIG. 8 is a block diagram showing a steering wheel detection device in which second sweat detection electrodes are disposed in spokes and that includes a plurality of sweat detection circuits.

FIG. 9 is a diagram showing: a rim in which each of the electrodes shown in FIG. 8 is disposed; and cross sections of the rim.

FIG. 10 is a block diagram showing a steering wheel detection device that includes a plurality of sweat detection circuits, a first switch, a second switch, and a switch controller.

FIG. 11 is a diagram showing: a rim in which each of the electrodes shown in FIG. 10 is disposed; and cross sections of the rim.

FIG. 12 is a block diagram showing a steering wheel detection device that includes a grip detection electrode and a grip detection circuit.

FIG. 13 is a diagram showing: a rim in which each of the electrodes shown in FIG. 12 is disposed; and cross sections of the rim.

FIG. 14 is a block diagram showing a steering wheel detection device that includes second heart rate detection electrodes disposed in spokes, a grip detection electrode, and a grip detection circuit.

FIG. 15 is a diagram showing: a rim in which each of the electrodes shown in FIG. 14 is disposed; and cross sections of the rim.

FIG. 16 is a block diagram showing a steering wheel detection device that includes a plurality of sweat detection circuits, a first switch, a second switch, a switch controller, a grip detection electrode, and a grip detection circuit.

FIG. 17 is a diagram showing: a rim in which each of the electrodes shown in FIG. 16 is disposed; and cross sections of the rim.

FIG. 18 is a diagram showing: a rim in which each of electrodes longer than the electrodes shown in each of FIG. 1A to FIG. 17 is disposed; and cross sections of the rim.

FIG. 19 is a block diagram showing a steering wheel detection device when a shield electrode is disposed in a rim.

FIG. 20 is a block diagram showing a steering wheel detection device when reference electrodes are disposed on the right side and the left side of a rim.

FIG. 21 is a diagram showing: the rim in which each of the electrodes shown in FIG. 20 is disposed; and cross sections of the rim.

### DESCRIPTION OF EMBODIMENT

Hereinafter, an embodiment is described in detail with reference to the Drawings.

It should be noted that the embodiment described below shows a general or specific example. The numerical values, shapes, materials, constituent elements, the arrangement and connection of the constituent elements, etc. shown in the following embodiment are mere examples, and are not intended to limit the present disclosure. Moreover, among the constituent elements in the following embodiment, those not recited in any one of the independent claims are described as optional constituent elements.

Furthermore, the figures each are a schematic diagram and are not necessarily an accurate illustration. In addition, in each figure, the same reference signs are assigned to the same constituent elements.

In the following embodiment, expressions such as T-shaped and right direction are used. For example, the expressions “T-shaped” and “right direction” not only mean exactly T-shaped and exactly right direction, but also mean substantially T-shaped and substantially right direction, allowing for an error margin of about several percent. More specifically, the expressions “T-shaped” and “right direction” mean T-shaped and right direction to an extent that the advantageous effects of the present disclosure can be achieved. The same applies to other expressions using “shaped” and “direction”.

### Embodiment

### Configuration

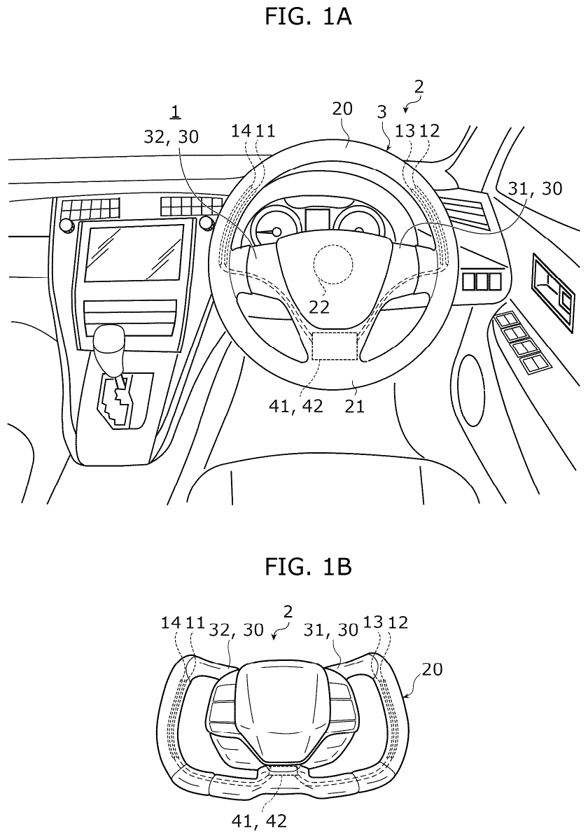

First, steering wheel detection device 3 provided in vehicle 1 is described with reference to FIG. 1A to FIG. 3.

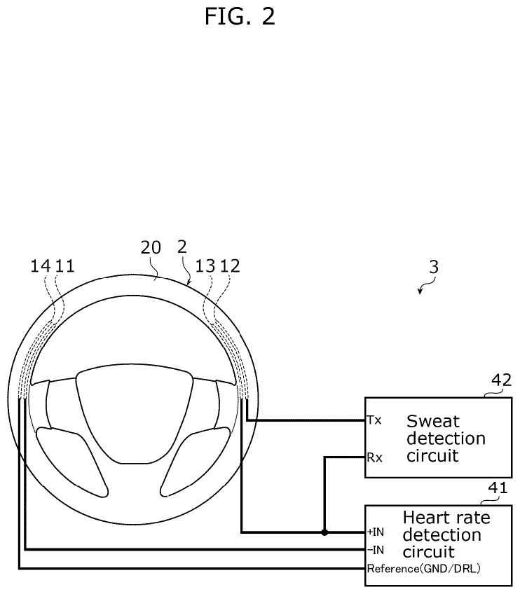

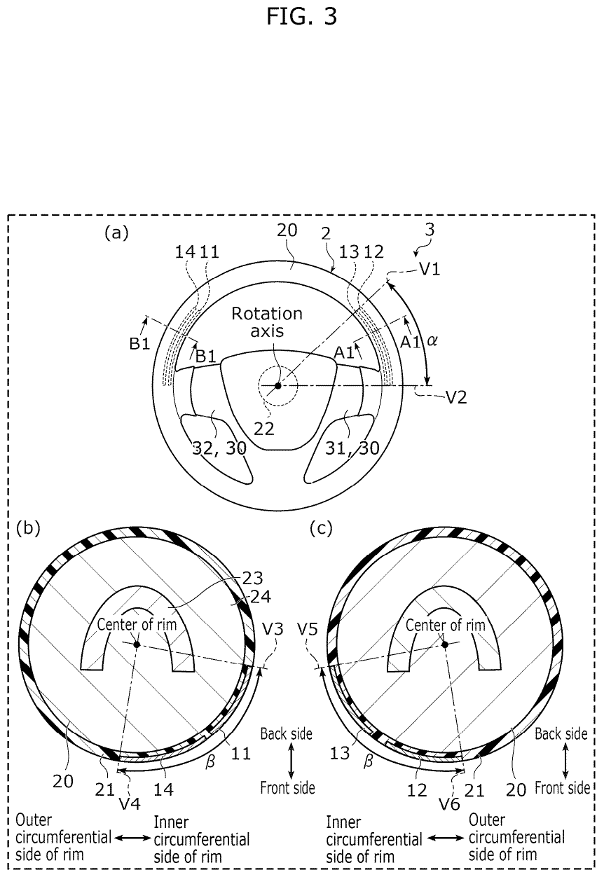

FIG. 1A is a schematic diagram showing an example of vehicle 1 in which steering wheel 2 according to an embodiment is provided. FIG. 1B is a front view of different steering wheel 2. FIG. 2 is a block diagram showing steering wheel detection device 3 according to the embodiment. FIG. 3 is a diagram showing: rim 20 in which each of electrodes that are first heart rate detection electrode 11, first sweat detection electrode 12, first heart rate and sweat detection electrode 13, and reference electrode 14 shown in FIG. 2 is disposed; and cross sections of rim 20. (a) of FIG. 3 shows rim 20 in which each of the electrodes shown in FIG. 2 is disposed and that is in the neutral position. (b) of FIG. 3 shows a cross section of rim 20 taken along line B1-B1 shown in (a) of FIG. 3. (c) of FIG. 3 shows a cross section of rim 20 taken along line A1-A1 shown in (a) of FIG. 3.

As shown in FIG. 1A, steering wheel 2 determines a steering angle when steering vehicle 1. Steering wheel 2 includes rim 20, spokes 30, and hub 22.

Rim 20 and spokes 30 are wrapped around by outer cover 21. Outer cover 21 includes: outer cover 21 that is wrapped around rim 20; and outer cover 21 that is wrapped around spokes 30. Outer cover 21 may be configured integral with rim 20 or may be detachable from rim 20.

Rim 20 is a grip portion to be gripped by a driver's (person's) hands. Rim 20 shown in FIG. 1A is of, for example, a ring type. It should be noted that rim 20 is not limited to a ring type and may be of an irregular type such as a yoke type shown in FIG. 1B, a D-type, and an oval type, though not shown.

As shown in FIG. 1A and FIG. 3, rim 20 includes core metal 23 and foam 24 that covers core metal 23.

Foam 24 constitutes the outer shell of rim 20 in steering wheel 2. Foam 24 includes a urethane resin material such as polyurethane. Core metal 23 is embedded in foam 24.

Spokes 30 are supporting members for connecting rim 20 and hub 22. Spokes 30 each include core metal 23 and foam 24 that covers core metal 23. Spokes 30 are disposed on the inner circumferential surface of rim 20. Spokes 30 are connected to rim 20 and form a T shape integrally with rim 20.

Hub 22 is a component for transmitting a rotational force generated by an operation on rim 20 to the steering shaft, and is located in the central portion of steering wheel 2.

As shown in FIG. 1A and FIG. 2, steering wheel detection device 3 is provided in steering wheel 2 of vehicle 1. Steering wheel detection device 3 makes it possible to obtain biological information about the driver who grips rim 20. The biological information about the driver includes: information that indicates the heart rate of the driver who grips rim 20; and information that indicates the sweating state of the driver who grips rim 20.

Specifically, steering wheel detection device 3 includes first heart rate detection electrode 11, first sweat detection electrode 12, first heart rate and sweat detection electrode 13, reference electrode 14, heart rate detection circuit 41, and sweat detection circuit 42. First heart rate detection electrode 11, first sweat detection electrode 12, first heart rate and sweat detection electrode 13, and reference electrode 14 are disposed in rim 20 of steering wheel 2 provided in vehicle 1.

First heart rate detection electrode 11, first sweat detection electrode 12, first heart rate and sweat detection electrode 13, and reference electrode 14 are disposed to be exposed on rim 20. Specifically, an opening for exposing each of first heart rate detection electrode 11, first sweat detection electrode 12, first heart rate and sweat detection electrode 13, and reference electrode 14 is provided in outer cover 21. Although outer cover 21 is wrapped around rim 20, outer cover 21 exposes each of first heart rate detection electrode 11, first sweat detection electrode 12, first heart rate and sweat detection electrode 13, and reference electrode 14.

In a plan view of rim 20 in the neutral position: first heart rate detection electrode 11 is disposed on one of the right side or the left side of rim 20; first heart rate and sweat detection electrode 13 is disposed on an other of the right side or the left side of rim 20; and first sweat detection electrode 12 is disposed adjacent to and not in contact with first heart rate and sweat detection electrode 13. In other words, in rim 20, first sweat detection electrode 12 is disposed on the same side as first heart rate and sweat detection electrode 13 and on an opposite side of first heart rate detection electrode 11.

More specifically, in the plan view of rim 20 in the neutral position: first heart rate detection electrode 11 is located on the right side of rim 20 to correspond to the right hand of the driver, and first heart rate and sweat detection electrode 13 and first sweat detection electrode 12 are located on the left side of rim 20 to correspond to the left hand of the driver; or first heart rate detection electrode 11 is located on the left side of rim 20 to correspond to the left hand of the driver, and first heart rate and sweat detection electrode 13 and first sweat detection electrode 12 are located on the right side of rim 20 to correspond to the right hand of the driver.

It should be noted that FIG. 1A to FIG. 3 each show an example in which first sweat detection electrode 12 and first heart rate and sweat detection electrode 13 are disposed on the right side of rim 20, and first heart rate detection electrode 11 and reference electrode 14 are disposed on the left side of rim 20. It should be noted that the right side or the left side of rim 20 in the plan view of rim 20 in the neutral position may be hereinafter simply referred to as the right side or the left side of rim 20, respectively.

Here, the expression “first sweat detection electrode 12 is disposed adjacent to” means, for example, that first sweat detection electrode 12 is disposed to cause a distance between first sweat detection electrode 12 and first heart rate and sweat detection electrode 13 to be less than the thickness of a driver's finger in order that, when the driver grips rim 20 with a driver's hand, the finger comes into contact with both first sweat detection electrode 12 and first heart rate and sweat detection electrode.

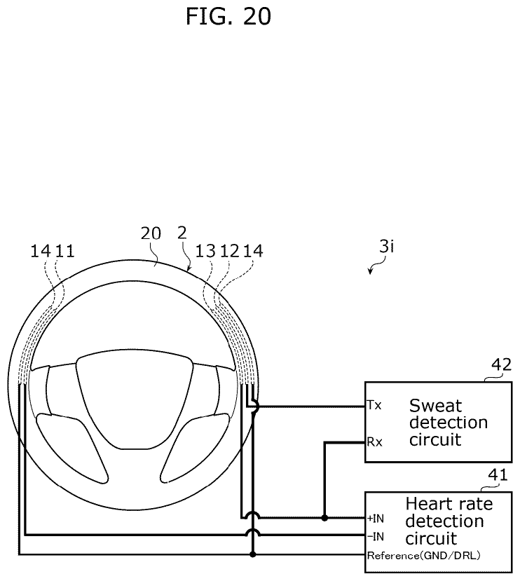

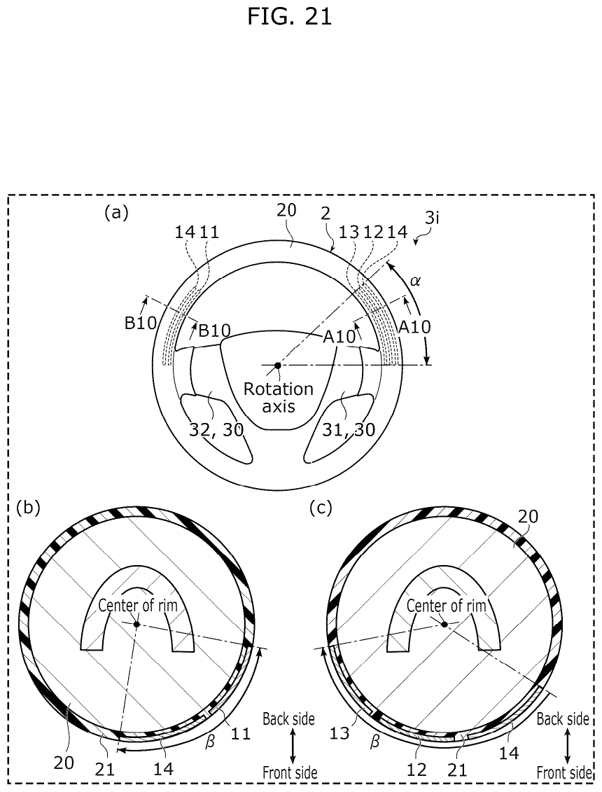

Reference electrode 14 is disposed on at least one of the right side or the left side of rim 20. Reference electrode 14 is disposed in rim 20, reference electrode 14 not being in contact with first heart rate detection electrode 11, first sweat detection electrode 12, or first heart rate and sweat detection electrode 13. It should be noted that a configuration in which reference electrodes 14 are disposed on both the right side and the left side of rim 20 is described with reference to FIG. 20 and FIG. 21 to be described later.

The surface area of reference electrode 14 may be set larger than the surface area of first heart rate detection electrode 11, the surface area of first sweat detection electrode 12, and the surface area of first heart rate and sweat detection electrode 13. Accordingly, when a driver's hand grips steering wheel 2, the driver's hand easily comes into contact with reference electrode 14.

As shown in (a) of FIG. 3, when the rotation axis of rim 20 (the axis of hub 22) is defined as a center, angle α formed by dash-dot straight line V1 and dash-dot straight line V2 is set to be at least 30° and at most 90°, dash-dot straight line V1 connecting the rotation axis and one end of each of first sweat detection electrode 12 and first heart rate and sweat detection electrode 13, dash-dot straight line V2 connecting the rotation axis and another end of each of first sweat detection electrode 12 and first heart rate and sweat detection electrode 13. It should be noted that angle α is at least 30° and at most 90°, and first heart rate detection electrode 11, first sweat detection electrode 12, first heart rate and sweat detection electrode 13, and reference electrode 14 are disposed in rim 20 in order that the electrodes disposed on the right side of rim 20 are not in contact with the electrodes disposed on the left side of rim 20.

As shown in (b) and (c) of FIG. 3, when the cross sections of rim 20 are viewed, first heart rate detection electrode 11, first sweat detection electrode 12, first heart rate and sweat detection electrode 13, and reference electrode 14 are disposed on the front side of rim 20 in vehicle 1.

For example, first heart rate and sweat detection electrode 13 and first sweat detection electrode 12 may be disposed on the front side of vehicle 1 in order that, when the driver grips steering wheel 2 with the right hand, the driver's right-hand fingers come into contact with first heart rate and sweat detection electrode 13 and first sweat detection electrode 12. In other words, first heart rate and sweat detection electrode 13 and first sweat detection electrode 12 are disposed in order that, when the driver grips steering wheel 2 with the right hand, the driver's right-hand fingers come into contact with both first heart rate and sweat detection electrode 13 and first sweat detection electrode 12.

Moreover, first heart detection electrode 11 and reference electrode 14 may be disposed on the front side of vehicle 1 in order that, when the driver grips steering wheel 2 with the left hand, the driver's left-hand fingers come into contact with first heart detection electrode 11 and reference electrode 14. To put it another way, first heart detection electrode 11 and reference electrode 14 are disposed in order that, when the driver grips steering wheel 2 with the left hand, the driver's left-hand fingers come into contact with both first heart detection electrode 11 and reference electrode 14.

When a cross section of rim 20 is circular, the front side of rim 20 is a sector having the central angle of 180°; however, regions in which the respective electrodes are disposed are set in order that angle β shown in (b) and (c) of FIG. 3 is in a range of at least 30° and at most 150°.

For example, in (b) of FIG. 3, reference electrode 14 and first heart rate detection electrode 11 that is located closer to the inner circumferential side of rim 20 than reference electrode 14 are disposed on the left side of rim 20, and angle β formed by dash-dot straight line V3 and dash-dot straight line V4 is set to be at least 30° and at most 150°, dash-dot straight line V3 connecting the center of the cross section of rim 20 (the center of rim 20) and one end of first heart rate detection electrode 11 located closer to the inner circumferential side of rim 20, dash-dot straight line V4 connecting the center of the cross section of rim 20 (the center of rim 20) and one end of reference electrode 14 located closer to the outer circumferential side of rim 20.

For example, in (c) of FIG. 3, first sweat detection electrode 12 and first heart rate and sweat detection electrode 13 that is located closer to the inner circumferential side of rim 20 than first sweat detection electrode 12 are disposed on the right side of rim 20, and angle β formed by dash-dot straight line V5 and dash-dot straight line V6 is set to be at least 30° and at most 150°, dash-dot straight line V5 connecting the center of the cross section of rim 20 (the center of rim 20) and one end of first heart rate and sweat detection electrode 13 located closer to the inner circumferential side of rim 20, dash-dot straight line V6 connecting the center of the cross section of rim 20 (the center of rim 20) and one end of first sweat detection electrode 12 located closer to the outer circumferential side of rim 20.

For example, in the present embodiment, when rim 20 in the neutral position is regarded as a clock, right-side spoke 30 extends from hub 22 in the three-o'clock direction, left-side spoke 30 extends from hub 22 in the nine-o'clock direction, and desired hand grip positions relative to rim 20 are an approximately three-o'clock position and an approximately nine-o'clock position (a position approximately between two-o'clock and four-o'clock and a position approximately between eight-o'clock and ten-o'clock) on rim 20. It is possible to dispose each of first heart rate detection electrode 11, first sweat detection electrode 12, first heart rate and sweat detection electrode 13, and reference electrode 14 at a corresponding one of the desired grip positions. For this reason, when the driver grips rim 20 with the hands at the approximately three-o'clock position and the approximately nine-o'clock position, the hands come into contact with first heart rate detection electrode 11, first sweat detection electrode 12, first heart rate and sweat detection electrode 13, and reference electrode 14. Accordingly, it is possible to stably obtain biological information about the driver.

First heart rate detection electrode 11, first sweat detection electrode 12, first heart rate and sweat detection electrode 13, and reference electrode 14 may be disposed in a front portion of rim 20 in vehicle 1, and at least one of first heart rate detection electrode 11, first sweat detection electrode 12, first heart rate and sweat detection electrode 13, or reference electrode 14 may be disposed closer to the outer circumferential side of rim 20.

Specifically, first heart rate and sweat detection electrode 13 is electrically connected to “+IN” of heart rate detection circuit 41, first heart rate detection electrode 11 is electrically connected to “−IN” of heart rate detection circuit 41, and reference electrode 14 is electrically connected to “Reference (GND/DRL)” of heart rate detection circuit 41. Each of the wires is covered with outer cover 21 to make the wire invisible from the outside of steering wheel 2.

Heart rate detection circuit 41 is a heart rate measurement device that detects the heart rate of the driver, based on heart rate signals outputted from each of first heart rate detection electrode 11 and first heart rate and sweat detection electrode 13 while the driver's hands come into contact with steering wheel 2. Heart rate signals outputted from first heart rate detection electrode 11, heart rate signals outputted from first heart rate and sweat detection electrode 13, and reference signals outputted from reference electrode 14 are inputted to heart rate detection circuit 41. Heart rate detection circuit 41 makes it possible to obtain information indicating the heart rate of the driver, by detecting the heart rate of the driver, based on signals outputted from each of first heart rate detection electrode 11, first heart rate and sweat detection electrode 13, and reference electrode 14.

Reference electrode 14 is connected to “GND” (Ground) or “DRL” (Driven Right Leg) of heart rate detection circuit 41, and makes it possible to supply a stable reference potential. The DRL is a noise cancellation circuit that inverts the phase of a noise signal inputted and outputs the noise signal to reference electrode 14 (the driver). Since reference electrode 14 supplies a reference potential, it is possible to measure, using the reference potential, a potential difference between first heart rate detection electrode 11 and first heart rate and sweat detection electrode 13 and a potential difference between first sweat detection electrode 12 and first heart rate and sweat detection electrode 13. For this reason, heart rate detection circuit 41 makes it possible to detect a heart rate obtained by reducing noise of heart rate signals obtained from first heart rate detection electrode 11 and first heart rate and sweat detection electrode 13, and sweat detection circuit 42 makes it possible to detect a sweating state obtained by reducing noise of sweat signals obtained from first sweat detection electrode 12 and first heart rate and sweat detection electrode 13.

Heart rate detection circuit 41 includes, for example, a low-pass filter, a differential amplifier, and an analog-to-digital converter. Heart rate detection circuit 41 inputs heart rate signals to an electronic control unit (ECU) provided in vehicle 1. Accordingly, the ECU makes it possible to obtain information indicating the heart rate of the driver. For example, the ECU makes it possible to cause an in-vehicle monitor provided in vehicle 1 to display the information indicating the heart rate of the driver. It should be noted that heart rate detection circuit 41 may cause the in-vehicle monitor to display the information indicating the heart rate of the driver indicated by the heart rate signals not via the ECU.

Sweat detection circuit 42 is electrically connected to first sweat detection electrode 12 and first heart rate and sweat detection electrode 13 via respective wires. Specifically, first sweat detection electrode 12 is electrically connected to “Tx” of sweat detection circuit 42, and first heart rate and sweat detection electrode 13 is electrically connected to “Rx” of sweat detection circuit 42 and “+IN” of heart rate detection circuit 41. Each of the wires is covered with outer cover 21 to make the wire invisible from the outside of steering wheel 2. Reference electrode 14 is electrically connected to heart rate detection circuit 41, and branches off from heart rate detection circuit 41 in parallel and is also electrically connected to sweat detection circuit 42 (not shown in the figures).

Sweat detection circuit 42 is a sweat detector that makes it possible to detect the sweating state of the driver's hands when the driver's hands come into contact with steering wheel 2. When sweat signals outputted from first sweat detection electrode 12 and first heart rate and sweat detection electrode 13 are inputted to sweat detection circuit 42, sweat detection circuit 42 makes it possible to detect the sweating state of the driver's hands.

Specifically, sweat detection circuit 42 includes a signal source that makes it possible to apply an alternating voltage to first sweat detection electrode 12. When the driver's hands grip rim 20 while the signal source is applying the alternating voltage to first sweat detection electrode 12, the driver's hands come into contact with first sweat detection electrode 12 and first heart rate and sweat detection electrode 13. For this reason, first sweat detection electrode 12 and first heart rate and sweat detection electrode 13 are electrically connected by the driver's hands. Since the signal source is applying the alternating voltage to first sweat detection electrode 12, a current flows from first sweat detection electrode 12 to first heart rate and sweat detection electrode 13 via the driver's hands, and a sweat signal outputted from first heart rate and sweat detection electrode 13 is inputted to sweat detection circuit 42. Sweat detection circuit 42 makes it possible to detect the sweating state of the driver's hands, based on the sweat signal outputted from first heart rate and sweat detection electrode 13.

Sweat detection circuit 42 includes, for example, a low-pass filter, a differential amplifier, and an analog-to-digital converter. Sweat detection circuit 42 inputs a sweat signal to the ECU provided in vehicle 1. Accordingly, the ECU makes it possible to obtain information indicating the sweating state of the driver. For example, the ECU makes it possible to cause the in-vehicle monitor provided in vehicle 1 to display the information indicating the sweating state of the driver. It should be noted that sweat detection circuit 42 may cause the in-vehicle monitor to display the information indicating the sweating state of the driver indicated by the sweat signal not via the ECU.

Heart rate detection circuit 41 and sweat detection circuit 42 detect a heart rate and a sweating state simultaneously, respectively. A configuration that separates the detection of a heart rate and the detection of a sweating state using a filter circuit (not shown in the figures) is used for this purpose. It should be noted that the present disclosure is not limited to a configuration that detects a heart rate and a sweating state simultaneously, and a configuration that detects a heart rate and a sweating state by time division using a switch not shown in the figures may be used. In other words, the detection of the heart rate and the detection of the sweating state may be performed mutually exclusively in order that a heart rate detection timing and a sweating state detection timing do not overlap temporally. Additionally, whether to perform the detection of the heart rate and the detection of the sweating state simultaneously or by time division may be selected according to a situation such as whether both data on the heart rate and data on the sweating state are always necessary or whether it is desired to detect both data on the heart rate and data on the sweating state sufficiently separately.

Steering wheel detection device 3 thus configured makes it possible to estimate an emotion of the driver, based on the heart rate and the sweating state of the driver. Examples of an emotion of the driver include emotions such as whether the driver is calm, nervous, or anxious.

The ECU may control in-vehicle equipment provided in vehicle 1, based on the emotion of the driver estimated by steering wheel detection device 3. Examples of the in-vehicle equipment include in-vehicle air conditioning equipment, in-vehicle audio equipment, and in-vehicle lighting equipment. For example, when the emotion of the driver is estimated to be nervous or anxious, the ECU may cause the in-vehicle equipment to alleviate the nervousness or the anxiety of the driver. For example, the ECU causes the in-vehicle air conditioning equipment to adjust a temperature, the in-vehicle lighting equipment to adjust, for example, a brightness and a color temperature, and the in-vehicle audio equipment to output music.

Steering wheel detection device 3 is expected to perform dead man detection. For example, although the driver is in traveling vehicle 1, the ECU may not detect the heart rate and the sweating state of the driver from steering wheel detection device 3 for at least a predetermined period. In this this, the ECU may switch vehicle 1 to autonomous driving, cause vehicle 1 to park by the side of a road, or send notification to emergency contacts (e.g., family, an automobile manufacturer, a hospital).

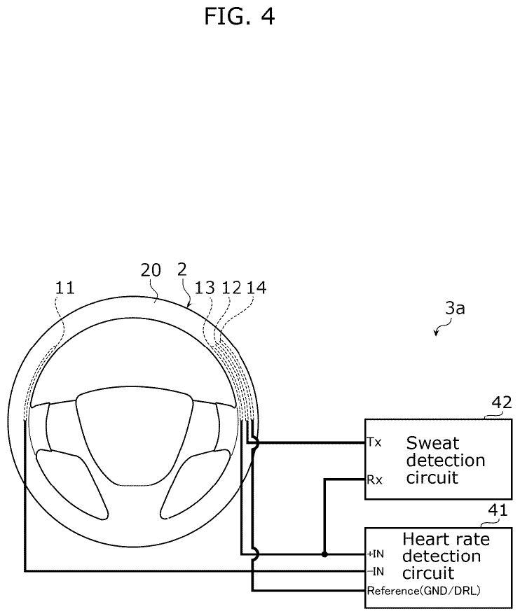

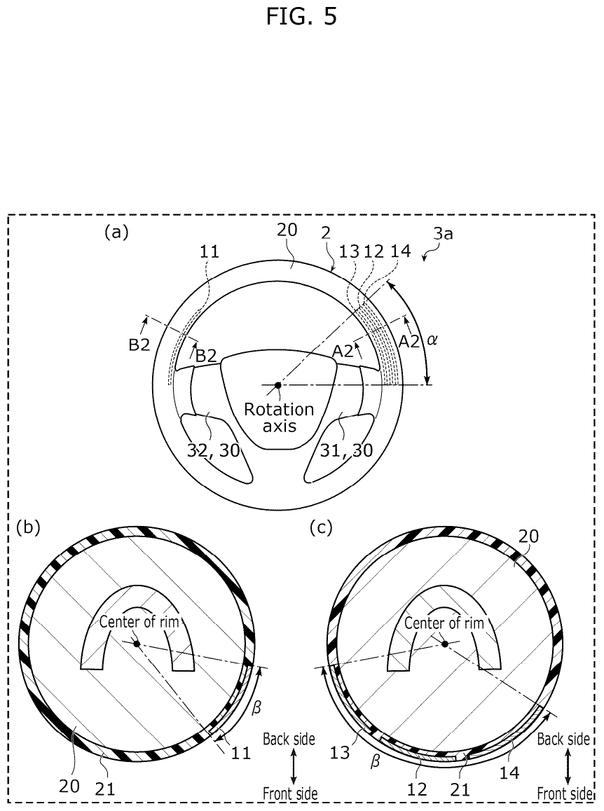

Although FIG. 1A to FIG. 3 show the example in which first sweat detection electrode 12 and first heart rate and sweat detection electrode 13 are disposed on the right side of rim 20, and first heart rate detection electrode 11 and reference electrode 14 are disposed on the left side of rim 20, the present disclosure is not limited to this example. For example, first sweat detection electrode 12, first heart rate and sweat detection electrode 13, and reference electrode 14 may be disposed on one of the right side or the left side of rim 20, and first heart rate detection electrode 11 may be disposed on an other of the right side or the left side of rim 20. FIG. 4 and FIG. 5 show an example of steering wheel detection device 3a in which first sweat detection electrode 12, first heart rate and sweat detection electrode 13, and reference electrode 14 are disposed on the right side of rim 20, and first heart rate detection electrode 11 is disposed on the left side of rim 20.

FIG. 4 is a block diagram showing steering wheel detection device 3a in which first sweat detection electrode 12, first heart rate and sweat detection electrode 13, and reference electrode 14 are disposed on one side of rim 20, and first heart rate detection electrode 11 is disposed on another side of rim 20. FIG. 5 is a diagram showing rim 20 in which each of the electrodes shown in FIG. 4 is disposed, and cross sections of rim 20. (a) of FIG. 5 shows rim 20 in which each of the electrodes shown in FIG. 4 is disposed and that is in the neutral position. (b) of FIG. 5 shows a cross section of rim 20 taken along line B2-B2 shown in (a) of FIG. 5. (c) of FIG. 5 shows a cross section of rim 20 taken along line A2-A2 shown in (a) of FIG. 5.

In rim 20, reference electrode 14 may be disposed on the same side as first sweat detection electrode 12 and first heart rate and sweat detection electrode 13. In this case, reference electrode 14 is disposed closer to the inner circumferential side or the outer circumferential side of rim 20 than first sweat detection electrode 12 and first heart rate and sweat detection electrode 13. Reference electrode 14 is not disposed between first sweat detection electrode 12 and first heart rate and sweat detection electrode 13.

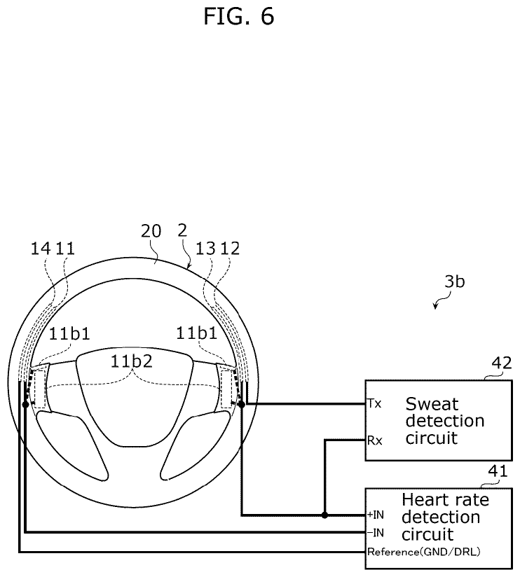

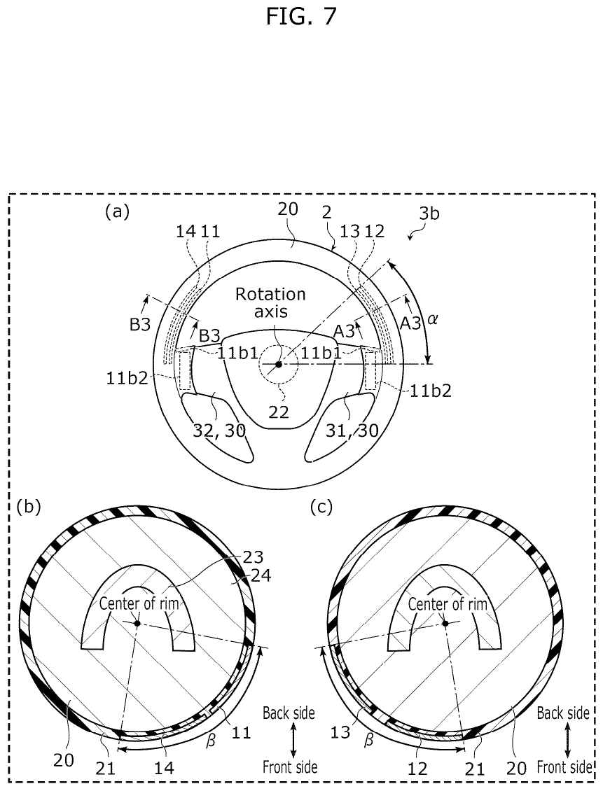

Although FIG. 1A to FIG. 5 show the example in which first heart rate detection electrode 11 is disposed in rim 20, the present disclosure is not limited to this example. For example, as shown in steering wheel detection device 3b in FIG. 6 and FIG. 7, second heart rate detection electrodes 11b1 and 11b 2 may be disposed in spokes 30.

FIG. 6 is a block diagram showing steering wheel detection device 3b in which second heart rate detection electrodes 11b1 and 11b2 are disposed in respective spokes 30. FIG. 7 is a diagram showing rim 20 in which each of the electrodes shown in FIG. 6 is disposed, and cross sections of rim 20. (a) of FIG. 7 shows rim 20 in which each of the electrodes shown in FIG. 6 is disposed and that is in the neutral position. (b) of FIG. 7 shows a cross section of rim 20 taken along line B3-B3 shown in (a) of FIG. 7. (c) of FIG. 7 shows a cross section of rim 20 taken along line A3-A3 shown in (a) of FIG. 7.

Steering wheel detection device 3b further includes second heart rate detection electrodes 11b1 and 11b2 disposed in spokes 30 of steering wheel 2.

Specifically, spokes 30 include first spoke 31 and second spoke 32.

When the driver views steering wheel 2 in the neutral position from front, first spoke 31 is disposed on the right side of steering wheel 2. When steering wheel 2 is in the neutral position, first spoke 31 is a portion closer to the right side than hub 22 and extends from hub 22 to rim 20 in the right direction.

When the driver views steering wheel 2 in the neutral position from front, second spoke 32 is disposed on the left side of steering wheel 2. When steering wheel 2 is in the neutral position, second spoke 32 is a portion closer to the left side than hub 22 and extends from hub 22 to rim 20 in the left direction.

For example, in the case where rim 20 when steering wheel 2 is in the neutral position is regarded as a clock, first spoke 31 extends from hub 22 in the three-o'clock direction, and second spoke 32 extends from hub 22 in the nine-o'clock direction. The neutral position is a rotational position when vehicle 1 is traveling straight and at which the front wheels of vehicle 1 correspond to a steering angle of zero.

When steering wheel 2 is in the neutral position, second heart rate detection electrode 11b1 may be disposed in each of an upper portion of the connection portion between first spoke 31 and rim 20 and an upper portion of the connection portion between second spoke 32 and rim 20. Second heart rate detection electrode 11b1 is disposed in each of first spoke 31 and second spoke 32 to be exposed on outer cover 21. Although outer cover 21 covers spokes 30, outer cover 21 includes openings for exposing second heart rate detection electrodes 11b1.

For example, second heart rate detection electrode 11b1 on the right side is disposed in first spoke 31 in order that, when the driver grips steering wheel 2 with the hands, second heart rate detection electrode 11b1 on the right side is brought into contact with a finger (e.g., the thumb or the little finger) of the right hand of the driver, and second heart rate detection electrode 11b1 on the left side is disposed in second spoke 32 in order that, when the driver grips steering wheel 2 with the hands, second heart rate detection electrode 11b1 on the left side is brought into contact with a finger (e.g., the thumb or the little finger) of the left hand of the driver.

Second heart rate detection electrode 11b2 may be disposed on each of: the front side of vehicle 1 in the vicinity of the connection portion between first spoke 31 and rim 20, that is, an opposite side of first spoke 31 closer to the driver; and the front side of vehicle 1 in the vicinity of the connection portion between second spoke 32 and rim 20, that is, an opposite side of second spoke 32 closer to the driver.

Second heart rate detection electrode 11b2 on the right side may be disposed in first spoke 31 on the front side of vehicle 1 in order that, when the driver grips steering wheel 2 with the hands, second heart rate detection electrode 11b2 on the right side is brought into contact with fingers (e.g., the middle finger, the ring finger, and the little finger) of the right hand of the driver, and second heart rate detection electrode 11b2 on the left side may be disposed in second spoke 32 on the front side of vehicle 1 in order that, when the driver grips steering wheel 2 with the hands, second heart rate detection electrode 11b2 on the left side is brought into contact with fingers (e.g., the middle finger, the ring finger, and the little finger) of the left hand of the driver.

Although FIG. 6 and FIG. 7 exemplify second heart rate detection electrodes 11b1 and 11b2 disposed in first spoke 31 and second spoke 32, respectively, the present disclosure is not limited to this example. For example, second heart rate detection electrodes 11b1 and 11b2 and reference electrode 14 may be disposed in spoke 30.

Instead of second heart rate detection electrode 11b1, reference electrode 14 may be disposed in the upper portion of the connection portion between first spoke 31 or second spoke 32 and rim 20. At this time, when reference electrode 14 is disposed in first spoke 31 or second spoke 32, reference electrode 14 may be disposed symmetrical to second heart rate detection electrode 11b1 about hub 22 of steering wheel 2.

Alternatively, instead of second heart rate detection electrode 11b2, reference electrode 14 may be disposed on the front side of vehicle 1 in the vicinity of the connection portion between first spoke 31 or second spoke 32 and rim 20, that is, the opposite side of first spoke 31 or second spoke 32 closer to the driver. At this time, when reference electrode 14 is disposed in spoke 30, reference electrode 14 may be disposed symmetrical to second heart rate detection electrode 11b2 about hub 22 of steering wheel 2.

Although FIG. 6 and FIG. 7 exemplify second heart rate detection electrodes 11b1 and 11b2 disposed in first spoke 31 and second spoke 32, respectively, the present disclosure is not limited to this example. For example, second heart rate detection electrodes 11b1 and 11b2 may be merely disposed in first spoke 31 or second spoke 32. In addition, second heart rate detection electrode 11b1 may be merely disposed in at least one of first spoke 31 or second spoke 32, and second heart rate detection electrode 11b2 may be merely disposed in at least one of first spoke 31 or second spoke 32.

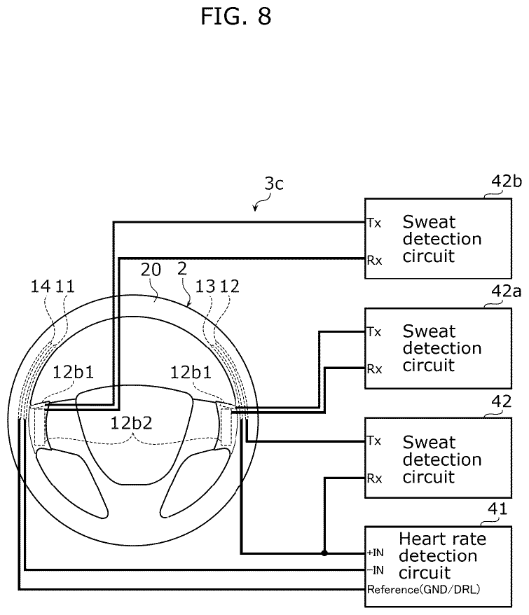

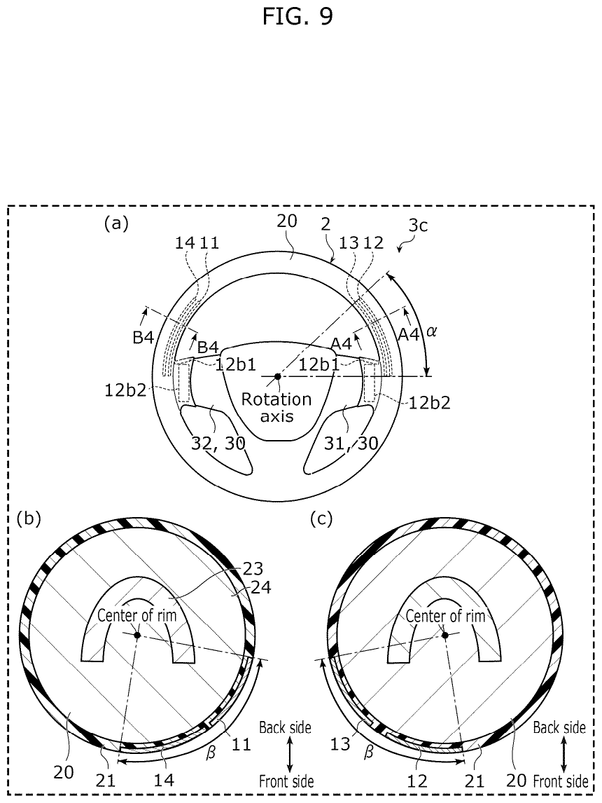

Although FIG. 6 and FIG. 7 exemplify second heart rate detection electrodes 11b1 and 11b2 disposed in spokes 30, the present disclosure is not limited to this example. For example, as shown in steering wheel detection device 3c in FIG. 8 and FIG. 9, instead of second heart rate detection electrodes 11b1 and 11b2, second sweat detection electrodes 12b1 and 12b2 may be disposed in spokes 30.

FIG. 8 is a block diagram showing steering wheel detection device 3c in which second sweat detection electrodes 12b1 and 12b2 are disposed in spokes 30 and that includes a plurality of sweat detection circuits 42. FIG. 9 is a diagram showing rim 20 in which each of the electrodes shown in FIG. 8 is disposed, and cross sections of rim 20. (a) of FIG. 9 shows rim 20 in which each of the electrodes shown in FIG. 8 is disposed and that is in the neutral position. (b) of FIG. 9 shows a cross section of rim 20 taken along line B4-B4 shown in (a) of FIG. 9. (c) of FIG. 9 shows a cross section of rim 20 taken along line A4-A4 shown in (a) of FIG. 9.

Steering wheel detection device 3c includes second sweat detection electrodes 12b1 and 12b2 disposed in spokes 30 of steering wheel 2.

When steering wheel 2 is in the neutral position, second sweat detection electrode 12b1 may be disposed in each of an upper portion of the connection portion between first spoke 31 and rim 20 and an upper portion of the connection portion between second spoke 32 and rim 20. Second sweat detection electrode 12b1 is disposed in each of first spoke 31 and second spoke 32 to be exposed on outer cover 21. Although outer cover 21 covers spokes 30, outer cover 21 includes openings for exposing second sweat detection electrodes 12b1.

For example, second sweat detection electrode 12b1 on the right side is disposed in first spoke 31 in order that, when the driver grips steering wheel 2 with the hands, second sweat detection electrode 12b1 on the right side is brought into contact with the thumb of the right hand of the driver, and second sweat detection electrode 12b1 on the left side is disposed in second spoke 32 in order that, when the driver grips steering wheel 2 with the hands, second sweat detection electrode 12b1 on the left side is brought into contact with the thumb of the left hand of the driver.

Second sweat detection electrode 12b2 may be disposed on each of: the front side of vehicle 1 in the vicinity of the connection portion between first spoke 31 and rim 20, that is, an opposite side of first spoke 31 closer to the driver; and the front side of vehicle 1 in the vicinity of the connection portion between second spoke 32 and rim 20, that is, an opposite side of second spoke 32 closer to the driver.

Second sweat detection electrode 12b2 on the right side may be disposed in first spoke 31 on the front side of vehicle 1 in order that, when the driver grips steering wheel 2 with the hands, second sweat detection electrode 12b2 on the right side is brought into contact with fingers (e.g., the middle finger, the ring finger, the index finger, and the little finger) of the right hand of the driver, and second sweat detection electrode 12b2 on the left side may be disposed in second spoke 32 on the front side of vehicle 1 in order that, when the driver grips steering wheel 2 with the hands, second sweat detection electrode 12b2 on the left side is brought into contact with fingers (e.g., the middle finger, the ring finger, the index finger, and the little finger) of the left hand of the driver.

Steering wheel detection device 3c may further include: sweat detection circuit 42a electrically connected to second sweat detection electrodes 12b1 and 12b2 disposed in first spoke 31; and sweat detection circuit 42b electrically connected to second sweat detection electrodes 12b1 and 12b2 disposed in second spoke 32. Sweat detection circuits 42, 42a, and 42b each include an identical configuration.

Although FIG. 8 and FIG. 9 exemplify second sweat detection electrodes 12b1 and 12b2 disposed in first spoke 31 and second spoke 32, respectively, the present disclosure is not limited to this example. For example, second sweat detection electrodes 12b1 and 12b2 may be merely disposed in first spoke 31 or second spoke 32. In this case, steering wheel detection device 3c may further include: sweat detection circuit 42a electrically connected to second sweat detection electrodes 12b1 and 12b2 disposed in first spoke 31; or sweat detection circuit 42b electrically connected to second sweat detection electrodes 12b1 and 12b2 disposed in second spoke 32.

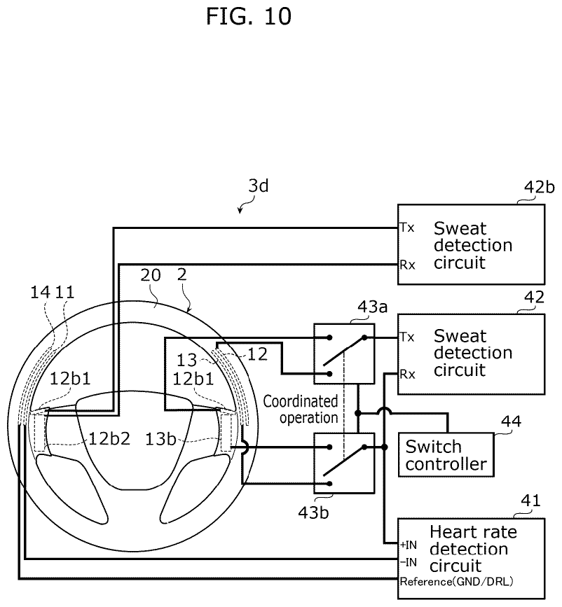

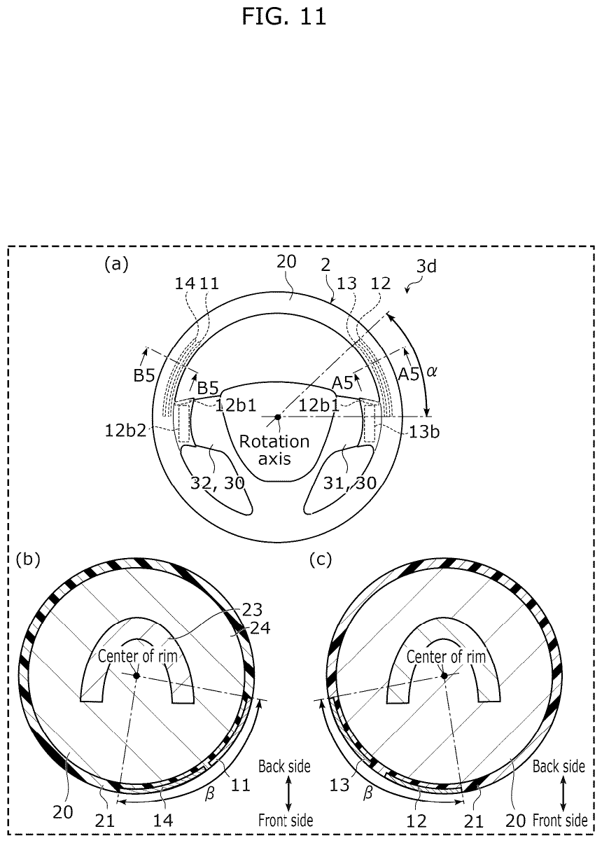

Although FIG. 8 and FIG. 9 exemplify first sweat detection electrode 12 and second sweat detection electrodes 12b1 and 12b2 disposed in spokes 30, the present disclosure is not limited to this example. As shown in steering wheel detection device 3d in FIG. 10 and FIG. 11, second sweat detection electrodes 12b1 and 12b2 and second heart rate and sweat detection electrode 13b may be disposed in spokes 30.

Since it is conceivable that the driver grips steering wheel 2 in various manners, it is possible to arrange electrodes according to how the driver grips steering wheel 2 (a way of gripping steering wheel 2).

FIG. 10 is a block diagram showing steering wheel detection device 3d that includes a plurality of sweat detection circuits 42, first switch 43a, second switch 43b, and switch controller 44. FIG. 11 is a diagram showing rim 20 in which each of the electrodes shown in FIG. 10 is disposed, and cross sections of rim 20. (a) of FIG. 11 shows rim 20 in which each of the electrodes shown in FIG. 10 is disposed and that is in the neutral position. (b) of FIG. 11 shows a cross section of rim 20 taken along line B5-B5 shown in (a) of FIG. 11. (c) of FIG. 11 shows a cross section of rim 20 taken along line A5-A5 shown in (a) of FIG. 11.

Steering wheel detection device 3d further includes second sweat detection electrodes 12b1 and 12b2 and second heart rate and sweat detection electrode 13b that are disposed in spokes 30 of steering wheel 2.

When steering wheel 2 is in the neutral position, second sweat detection electrode 12b1 may be disposed in each of an upper portion of the connection portion between first spoke 31 and rim 20 and an upper portion of the connection portion between second spoke 32 and rim 20. Second sweat detection electrode 12b1 is disposed in each of first spoke 31 and second spoke 32 to be exposed on outer cover 21. Although outer cover 21 covers spokes 30, outer cover 21 includes openings for exposing second sweat detection electrodes 12b1.

For example, second sweat detection electrode 12b1 on the right side is disposed in first spoke 31 in order that, when the driver grips steering wheel 2 with the hands, second sweat detection electrode 12b1 on the right side is brought into contact with the thumb of the right hand of the driver, and second sweat detection electrode 12b1 on the left side is disposed in second spoke 32 in order that, when the driver grips steering wheel 2 with the hands, second sweat detection electrode 12b1 on the left side is brought into contact with the thumb of the left hand of the driver.

Second sweat detection electrode 12b2 may be disposed on: the front side of vehicle 1 in the vicinity of the connection portion between first spoke 31 and rim 20, that is, an opposite side of first spoke 31 closer to the driver; or the front side of vehicle 1 in the vicinity of the connection portion between second spoke 32 and rim 20, that is, an opposite side of second spoke 32 closer to the driver.

Second heart rate and sweat detection electrode 13b is disposed symmetrical to second sweat detection electrode 12b2 about hub 22 of steering wheel 2. In other words, second heart rate and sweat detection electrode 13b may be disposed on: the front side of vehicle 1 in the vicinity of the connection portion between second spoke 32 and rim 20, that is, the opposite side of second spoke 32 closer to the driver; or the front side of vehicle 1 in the vicinity of the connection portion between first spoke 31 and rim 20, that is, the opposite side of first spoke 31 closer to the driver.

Although FIG. 10 and FIG. 11 show the example in which first heart rate and sweat detection electrode 13 is disposed on the right side of rim 20, and second heart rate and sweat detection electrode 13b is disposed in first spoke 31, the present disclosure is not limited to this example. For example, first heart rate and sweat detection electrode 13 and second heart rate and sweat detection electrode 13b may be disposed on the left side of rim 20. To put it another way, both first heart rate and sweat detection electrode 13 and second heart rate and sweat detection electrode 13b are disposed on the right side or the left side of steering wheel 2.

For example, first heart rate and sweat detection electrode 13, second heart rate and sweat detection electrode 13b, and first sweat detection electrode 12 may be disposed on the front side of vehicle 1 in order that, when the driver grips steering wheel 2 with the right hand, the right hand (e.g., the palm, the middle finger, the ring finger, the index finger, the little finger) of the driver comes into contact with first heart rate and sweat detection electrode 13, second heart rate and sweat detection electrode 13b, and first sweat detection electrode 12. In other words, first heart rate and sweat detection electrode 13, second heart rate and sweat detection electrode 13b, and first sweat detection electrode 12 are disposed in order that, when the driver grips steering wheel 2 with the hands, the right hand of the driver comes into contact with both second heart rate and sweat detection electrode 13b and first heart rate and sweat detection electrode 13 and further with both first heart rate and sweat detection electrode 13 and first sweat detection electrode 12. In this case, switch controller 44 to be described later performs switching to cause first switch 43a and second switch 43b to select first sweat detection electrode 12 and first heart rate and sweat detection electrode 13, respectively. Accordingly, sweat detection circuit 42 makes it possible to detect sweating of the right hand of the driver.

First heart rate detection electrode 11, reference electrode 14, and second sweat detection electrodes 12b1 and 12b2 may be disposed on the front side of vehicle 1 in order that, when the driver grips steering wheel 2 with the left hand, the left hand (e.g., the thumb, the palm, the middle finger, the ring finger, the index finger, the little finger) of the driver comes into contact with first heart rate detection electrode 11, reference electrode 14, and second sweat detection electrodes 12b1 and 12b2. To put it differently, first heart rate detection electrode 11, reference electrode 14, and second sweat detection electrodes 12b1 and 12b2 are disposed in order that, when the driver grips steering wheel 2 with the hands, the left hand of the driver comes into contact with both second sweat detection electrode 12b1 and second sweat detection electrode 12b2 by the left thumb and left fingertips of the driver coming into contact with second sweat detection electrode 12b1 and second sweat detection electrode 12b2, respectively. Accordingly, sweat detection circuit 42b makes it possible to detect sweating of the left hand of the driver.

Steering wheel detection device 3d may further include first switch 43a, second switch 43b, and switch controller 44.

First switch 43a is electrically connected to first sweat detection electrode 12, second sweat detection electrode 12b1, and sweat detection circuit 42. By being controlled by switch controller 44, first switch 43a switches between electrically connecting first sweat detection electrode 12 to sweat detection circuit 42 and electrically connecting second sweat detection electrode 12b1 to sweat detection circuit 42.

Second switch 43b is electrically connected to first heart rate and sweat detection electrode 13, second heart rate and sweat detection electrode 13b, sweat detection circuit 42, and heart rate detection circuit 41. By being controlled by switch controller 44, second switch 43b switches between electrically connecting first heart rate and sweat detection electrode 13 to sweat detection circuit 42 and heart rate detection circuit 41 and electrically connecting second heart rate and sweat detection electrode 13b to sweat detection circuit 42 and heart rate detection circuit 41.

Switch controller 44 causes first switch 43a and second switch 43b to switch in a coordinated manner between (i) electrically connecting first heart rate and sweat detection electrode 13 to sweat detection circuit 42 and heart rate detection circuit 41 while electrically connecting first sweat detection electrode 12 to sweat detection circuit 42 (a first connection state) and (ii) electrically connecting second heart rate and sweat detection electrode 13b to sweat detection circuit 42 and heart rate detection circuit 41 while electrically connecting second sweat detection electrode 12b1 to sweat detection circuit 42 (a second connection state).

For example, when it is impossible to obtain a sweat signal and a heart rate signal in the first connection state, switch controller 44 controls first switch 43a and second switch 43b to achieve the second connection state. Additionally, when it is impossible to obtain a sweat signal and a heart rate signal in the second connection state, switch controller 44 controls first switch 43a and second switch 43b to achieve the first connection state.

Although FIG. 10 and FIG. 11 exemplify second sweat detection electrode 12b1 and second heart rate and sweat detection electrode 13b disposed in first spoke 31, and second sweat detection electrodes 12b1 and 12b2 disposed in second spoke 32, the present disclosure is not limited to this example. For example, second sweat detection electrodes 12b1 and 12b2 need not be disposed in second spoke 32, and second sweat detection electrode 12b1 and second heart rate and sweat detection electrode 13b may be disposed in first spoke 31.

In addition, second sweat detection electrode 12b1 and second heart rate and sweat detection electrode 13b may be disposed in second spoke 32. At this time, first sweat detection electrode 12 and first heart rate and sweat detection electrode 13 may be disposed on the left side of rim 20, and first heart rate detection electrode 11 may be disposed on the right side of rim 20.

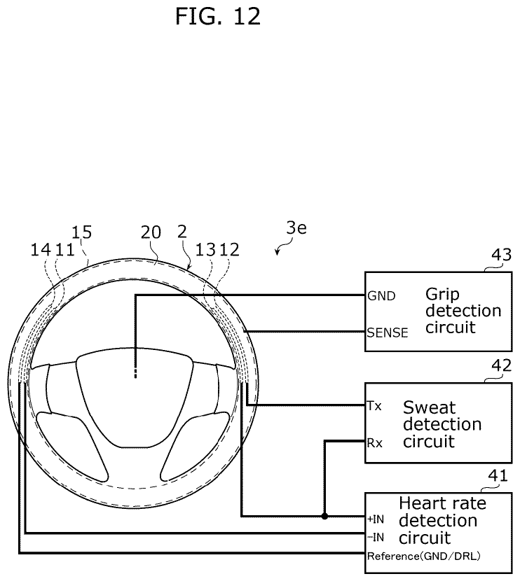

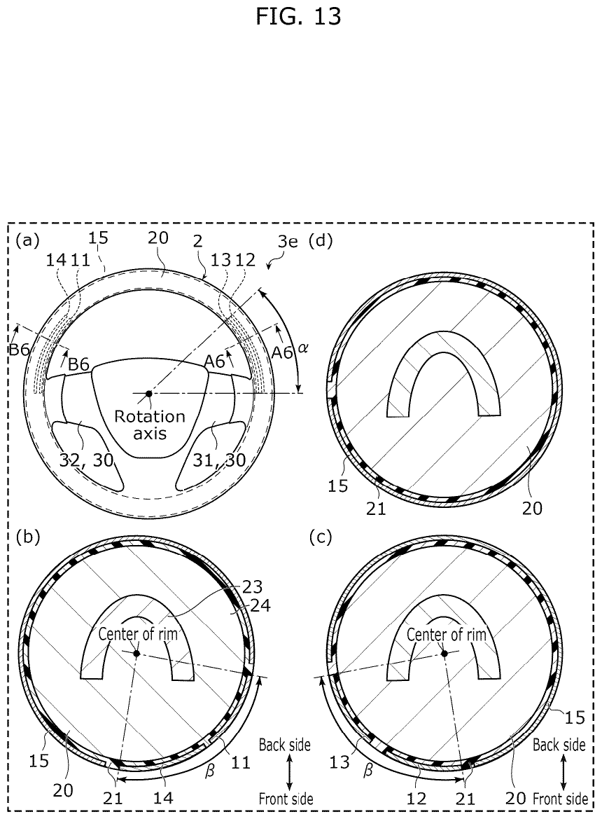

Although FIG. 1A to FIG. 11 exemplify first heart rate detection electrode 11, first sweat detection electrode 12, first heart rate and sweat detection electrode 13, reference electrode 14, sweat detection circuit 42, and heart rate detection circuit 41, the present disclosure is not limited to these examples. For example, as shown in FIG. 12 and FIG. 13, steering wheel detection device 3e may further include grip detection electrode 15 and grip detection circuit 43.

FIG. 12 is a block diagram showing steering wheel detection device 3e that includes grip detection electrode 15 and grip detection circuit 43. FIG. 13 is a diagram showing rim 20 in which each of the electrodes shown in FIG. 12 is disposed, and cross sections of rim 20. (a) of FIG. 13 shows rim 20 in which each of the electrodes shown in FIG. 12 is disposed and that is in the neutral position. (b) of FIG. 13 shows a cross section of rim 20 taken along line B6-B6 shown in (a) of FIG. 13. (c) of FIG. 13 shows a cross section of rim 20 taken along line A6-A6 shown in (a) of FIG. 13. (d) of FIG. 13 shows a cross section of rim 20 in a portion in which only grip detection electrode 15 is disposed.

Grip detection electrode 15 is disposed in rim 20, grip detection electrode 15 not being in contact with first heart rate detection electrode 11, first sweat detection electrode 12, first heart rate and sweat detection electrode 13, or reference electrode 14.

When a cross section of rim 20 is circular, grip detection electrode 15 is disposed along rim 20 except for, for example, a sector having a central angle of angle β (subtracting 150° from 360°) in cross section A6-A6 and cross section B6-B6 of rim 20, to have a central angle larger than the minimum value (30°) of angles α and β, that is, a central angle that is larger than 30° and smaller than or equal to 210°. In the portion in which only grip detection electrode 15 is disposed, grip detection electrode 15 is disposed along rim 20 to have a central angle that is larger than 30° and smaller than or equal to 360°.

Grip detection electrode 15 is a sensor electrode that is provided inside outer cover 21 wrapped around rim 20 and detects contact of a hand of the driver with outer cover 21. Grip detection electrode 15 is disposed in the circumferential direction of rim 20 in an annular shape. It should be noted that grip detection electrode 15 may be divided and disposed along the entire circumference of rim 20. For example, one grip detection electrode 15 may be disposed on the right side of rim 20, and another grip detection electrode 15 may be disposed on the left side of rim 20. Here, the term “contact” means not only that a hand of the driver comes into direct contact with outer cover 21, but also that, as long as grip detection electrode 15 makes it possible to detect a hand of the driver, a hand of the driver comes into indirect contact with outer cover 21 via an object and is spaced apart from outer cover 21.

Grip detection electrode 15 is electrically connected to “SENSE” of grip detection circuit 43. When grip detection electrode 15 detects contact of a hand of the driver with outer cover 21, grip detection electrode 15 inputs a detection signal to grip detection circuit 43.

Grip detection circuit 43 is embedded in, for example, spoke 30. Grip detection circuit 43 is a grip detector that detects contact of a hand of the driver with outer cover 21, based on each of detection signals outputted from grip detection electrode 15. Grip detection circuit 43 makes it possible to detect whether a hand of the driver comes into contact with outer cover 21, based on a change in capacitance between the hand of the driver and grip detection electrode 15. In addition, grip detection circuit 43 also makes it possible to detect a contact position of a hand of the driver, using a plurality of grip detection electrodes 15.

Specifically, grip detection circuit 43 applies an alternating voltage to grip detection electrode 15. At this time, when a hand of the driver comes into contact with the surface of outer cover 21, the capacitance of grip detection electrode 15 corresponding to the contact portion changes. For this reason, grip detection circuit 43 measures a change in capacitance in grip detection electrode 15, based on the voltage value of grip detection electrode 15. For example, when the hand of the driver is spaced apart from outer cover 21, grip detection circuit 43 detects a capacitance between vehicle 1 and grip detection electrode 15. Additionally, when the hand of the driver comes close to or into contact with outer cover 21, the capacitance changes because the hand of the driver is located between grip detection electrode 15 and the vehicle body. When the detected capacitance reaches at least a defined value, it is possible to determine that the hand of the driver is in contact with or grips outer cover 21.

Grip detection circuit 43 outputs a detection result according to a detection signal detected by each of grip detection electrodes 15. For example, grip detection circuit 43 inputs, to an ECU etc., a detection result indicating that a hand of the driver is in contact with outer cover 21.

Grip detection circuit 43 is grounded by being electrically connected to core metal 23 constituting steering wheel 2.

To detect contact of a hand of the driver with outer cover 21 and detect the sweating state of the hand of the driver, an alternating voltage supplied to grip detection electrode 15 and an alternating voltage supplied to first sweat detection electrode 12 are configured to differ in frequency.

Steering wheel detection device 3e thus configured is expected to perform dead man detection. For example, although the driver is in traveling vehicle 1, an ECU may not obtain detection of a heart rate, detection of sweat, and detection of a grip from steering wheel detection device 3e for at least a predetermined period. In this this, the ECU may switch vehicle 1 to autonomous driving, cause vehicle 1 to park by the side of a road, or send notification to emergency contacts (e.g., family, an automobile manufacturer, a hospital).

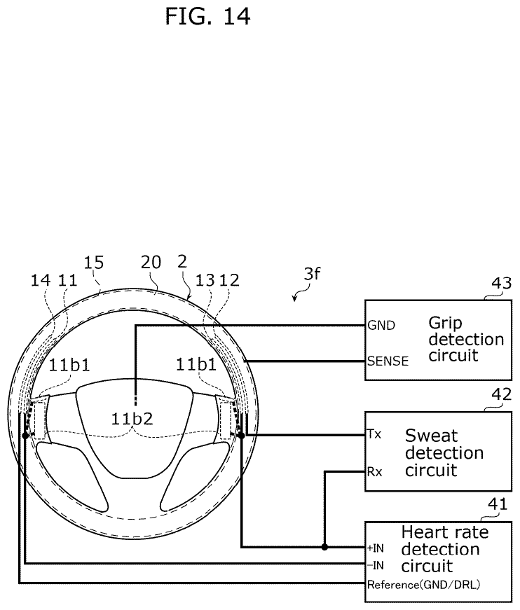

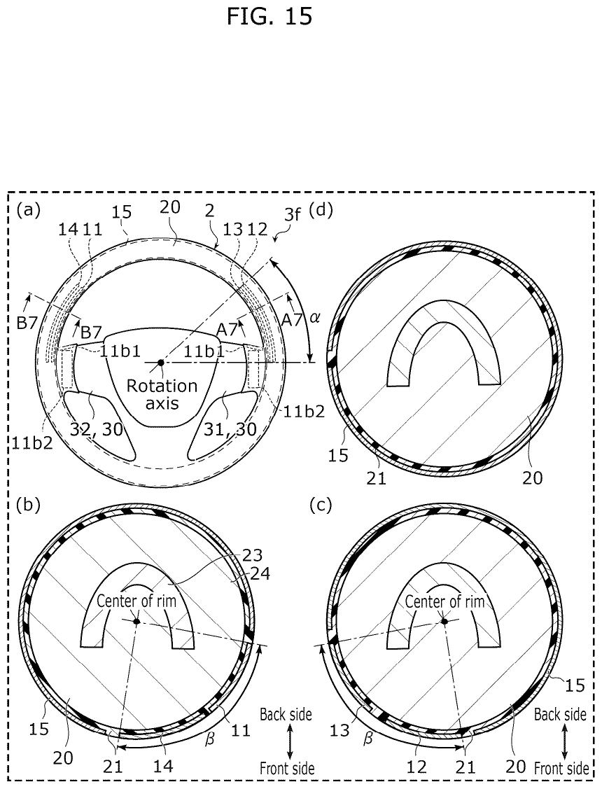

Although FIG. 6 and FIG. 7 show the example in which first heart rate detection electrode 11 is disposed in rim 20, and second heart rate detection electrodes 11b1 and 11b2 are disposed in spokes 30, the present disclosure is not limited to this example. As shown in FIG. 14 and FIG. 15, steering wheel detection device 3f may further include grip detection electrode 15 and grip detection circuit 43 in addition to the above configuration. Since grip detection electrode 15 and grip detection circuit 43 include configurations identical to those shown in FIG. 12 and FIG. 13, the descriptions thereof are omitted.

FIG. 14 is a block diagram showing steering wheel detection device 3f that includes second heart rate detection electrodes 11b1 and 11b2 disposed in spokes 30, grip detection electrode 15, and grip detection circuit 43. FIG. 15 is a diagram showing rim 20 in which each of the electrodes shown in FIG. 14 is disposed, and cross sections of rim 20. (a) of FIG. 15 shows rim 20 in which each of the electrodes shown in FIG. 14 is disposed and that is in the neutral position. (b) of FIG. 15 shows a cross section of rim 20 taken along line B7-B7 shown in (a) of FIG. 15. (c) of FIG. 15 shows a cross section of rim 20 taken along line A7-A7 shown in (a) of FIG. 15. (d) of FIG. 15 shows a cross section of rim 20 in a portion in which only grip detection electrode 15 is disposed.

It should be noted that, in the configuration shown in FIG. 14 and FIG. 15, as shown by the thick solid line and the thick dashed line in FIG. 14, second heart rate detection electrodes 11b1 and 11b2 disposed in first spoke 32 and first heart rate detection electrode 11 are electrically connected to “−IN” of heart rate detection circuit 41 using wires of second heart rate detection electrodes 11b1 and 11b2 and first heart rate detection electrode 11 as one wire. As shown by the thick solid line and the thick dashed line in FIG. 14, second heart rate detection electrodes 11b1 and 11b2 disposed in second spoke 31 and first heart rate and sweat detection electrode 13 are also electrically connected to “+IN” of heart rate detection circuit 41 using wires of second heart rate detection electrodes 11b1 and 11b2 and first heart rate and sweat detection electrode 13 as one wire. Since such wires make it possible to detect the heart rate of the driver using at least one of the three electrodes connected to “−IN” or at least one of the three electrodes connected to “+IN”, it is possible to increase a degree of freedom of a grip position of the driver.

Although FIG. 8 and FIG. 9 show the example in which first sweat detection electrode 12 is disposed in rim 20, and second sweat detection electrodes 12b1 and 12b2 are disposed in spokes 30, the present disclosure is not limited to this example. Steering wheel detection device 3c shown in FIG. 8 and FIG. 9 may further include grip detection electrode 15 and grip detection circuit 43 in addition to the above configuration. Since grip detection electrode 15 and grip detection circuit 43 include configurations identical to those shown in FIG. 12 and FIG. 13, the descriptions thereof are omitted.

In the above case, steering wheel detection device 3c shown in FIG. 8 and FIG. 9 may further include: sweat detection circuit 42a electrically connected to second sweat detection electrodes 12b1 and 12b2 disposed in first spoke 31; and sweat detection circuit 42b electrically connected to second sweat detection electrodes 12b1 and 12b2 disposed in second spoke 32.

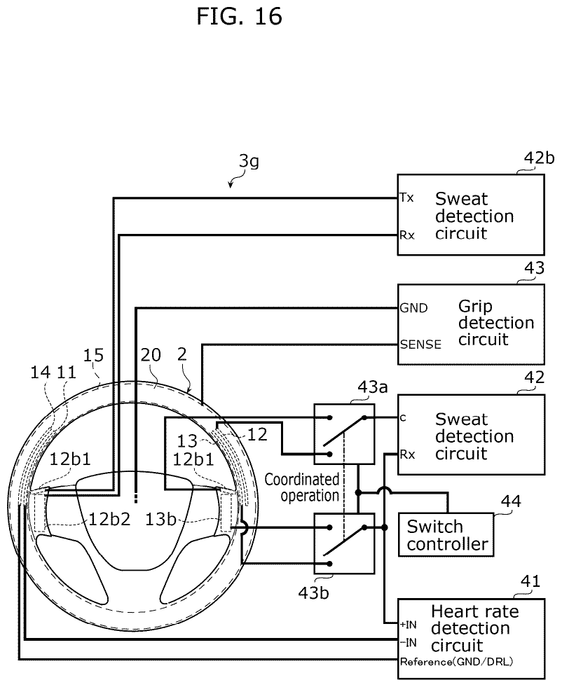

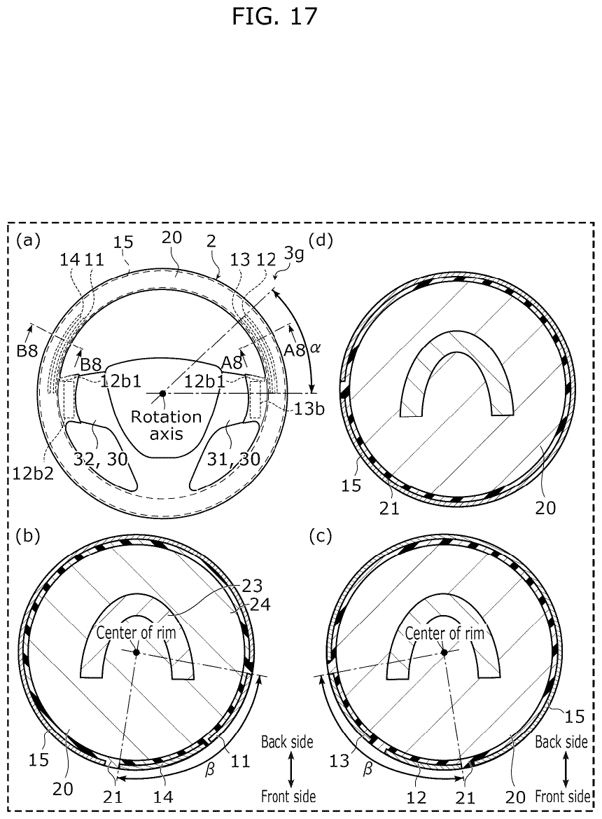

Although FIG. 10 and FIG. 11 show the example in which first heart rate and sweat detection electrode 13 is disposed in rim 20, and second heart rate and sweat detection electrode 13b is disposed in spoke 30 on the same side as first heart rate and sweat detection electrode 13, the present disclosure is not limited to this example. As shown in FIG. 16 and FIG. 17, steering wheel detection device 3g may further include grip detection electrode 15 and grip detection circuit 43 in addition to the above configuration. Since grip detection electrode 15 and grip detection circuit 43 include configurations identical to those shown in FIG. 12 and FIG. 13, the descriptions thereof are omitted.

FIG. 16 is a block diagram showing steering wheel detection device 3g that includes a plurality of sweat detection circuits 42 and 42b, first switch 43a, second switch 43b, switch controller 44, grip detection electrode 15, and grip detection circuit 43. FIG. 17 is a diagram showing rim 20 in which each of the electrodes shown in FIG. 16 is disposed, and cross sections of rim 20. (a) of FIG. 17 shows rim 20 in which each of the electrodes shown in FIG. 16 is disposed and that is in the neutral position. (b) of FIG. 17 shows a cross section of rim 20 taken along line B8-B8 shown in (a) of FIG. 17. (c) of FIG. 17 shows a cross section of rim 20 taken along line A8-A8 shown in (a) of FIG. 17. (d) of FIG. 17 shows a cross section of rim 20 in a portion in which only grip detection electrode 15 is disposed.

In this case, as with FIG. 10 and FIG. 11, steering wheel detection device 3g shown in FIG. 16 may further include sweat detection circuit 42b that is electrically connected to second sweat detection electrodes 12b1 and 12b2 disposed in second spoke 32, first switch 43a, second switch 43b, and switch controller 44.

Although second heart rate detection electrodes 11b1 and 11b2 disposed in first spoke 31 and second spoke 32, respectively, are exemplified in steering wheel detection device 3f shown in FIG. 14 and FIG. 15, the present disclosure is not limited to this example.

For example, instead of second heart rate detection electrodes 11b1 and 11b2, second sweat detection electrodes 12b1 and 12b2 may be disposed in spokes 30. In this case, second sweat detection electrodes 12b1 and 12b2 may be disposed in at least one of first spoke 31 or second spoke 32. Additionally, steering wheel detection device 3f may further include at least one of sweat detection circuit 42a electrically connected to second sweat detection electrodes 12b1 and 12b2 disposed in spoke 31 or sweat detection circuit 42b electrically connected to second sweat detection electrodes 12b1 and 12b2 disposed in second spoke 32.

Although FIG. 16 and FIG. 17 exemplify second sweat detection electrode 12b1 and second heart rate and sweat detection electrode 13b disposed in first spoke 31, and second sweat detection electrodes 12b1 and 12b2 disposed in second spoke 32, the present disclosure is not limited to this example.

For example, second sweat detection electrodes 12b1 and 12b2 need not be disposed in second spoke 32. In this case, second sweat detection electrode 12b1 and second heart rate and sweat detection electrode 13b may be disposed in second spoke 32. At this time, at least first sweat detection electrode 12 and first heart rate and sweat detection electrode 13 may be disposed on the left side of rim 20, and at least first heart rate detection electrode 11 may be disposed on the right side of rim 20.

Although FIG. 16 and FIG. 17 exemplify a first set of second sweat detection electrode 12b1 and second heart rate and sweat detection electrode 13b disposed in first spoke 31, and a second set of second sweat detection electrodes 12b1 and 12b2 disposed in second spoke 32, the present disclosure is not limited to this example.

For example, the second set of second sweat detection electrodes 12b1 and 12b2 may be disposed in first spoke 31 or second spoke 32, and the first set of second sweat detection electrode 12b1 and second heart rate and sweat detection electrode 13b need not be disposed in spoke 30. In this case, steering wheel detection device 3g may further include: sweat detection circuit 42a electrically connected to second sweat detection electrodes 12b1 and 12b2 disposed in first spoke 31; or sweat detection circuit 42b electrically connected to second sweat detection electrodes 12b1 and 12b2 disposed in second spoke 32.

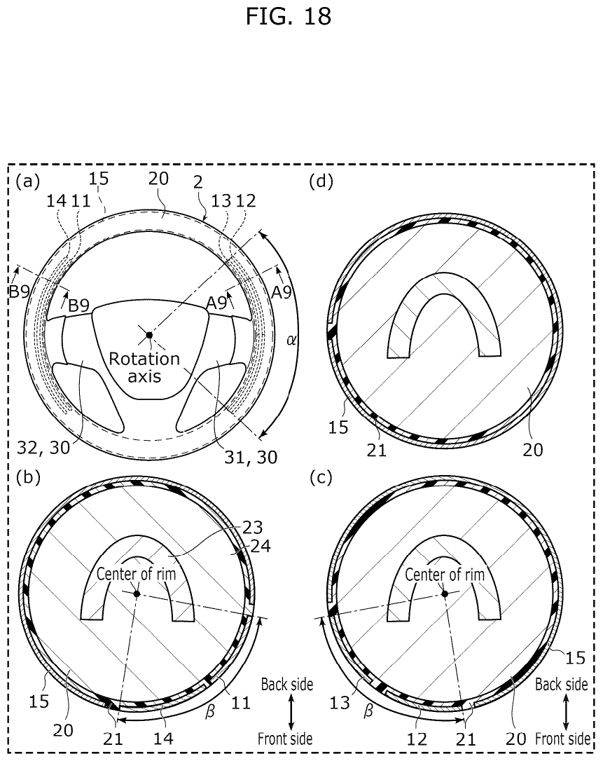

As shown in FIG. 18, first heart rate detection electrode 11, first sweat detection electrode 12, first heart rate and sweat detection electrode 13, and reference electrode 14 are disposed in the circumferential direction of rim 20, and these electrodes may be longer than the above-described respective electrodes. FIG. 18 is a diagram showing: rim 20 in which each of the electrodes longer than the respective electrodes shown in each of FIG. 1A to FIG. 17 is disposed; and cross sections of rim 20. (a) of FIG. 18 shows rim 20 in which each of the electrodes shown in FIG. 1A to FIG. 17 is disposed and that is in the neutral position, and exemplifies first heart rate detection electrode 11, first sweat detection electrode 12, first heart rate and sweat detection electrode 13, and reference electrode 14 when angle α is 90°. (b) of FIG. 18 shows a cross section of rim 20 taken along line B9-B9 shown in (a) of FIG. 18. (c) of FIG. 18 shows a cross section of rim 20 taken along line A9-A9 shown in (a) of FIG. 18. (d) of FIG. 18 shows a cross section of rim 20 in a portion in which only grip detection electrode 15 is disposed.

Although steering wheel detection devices 3e to 3g each further include grip detection electrode 15 and grip detection circuit 43 in FIG. 12 to FIG. 18, the present disclosure is not limited to these examples.

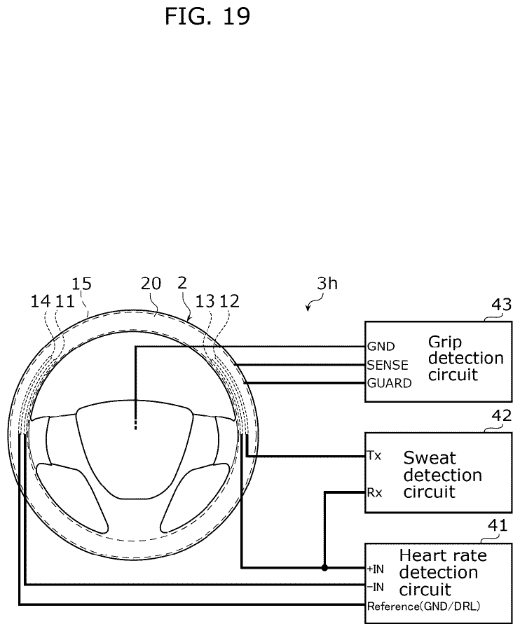

For example, as shown in FIG. 19, steering wheel detection device 3h may further include a shield electrode. FIG. 19 is a block diagram showing steering wheel detection device 3h when a shield electrode is disposed in rim 20.

The shield electrode is disposed between grip detection electrode 15 and core metal 23 (not shown in FIG. 19) of rim 20, and is electrically connected to “GUARD” of grip detection circuit 43. Accordingly, it is possible to reduce generation of a capacitance between grip detection electrode 15 and core metal 23 of rim 20. For this reason, it is possible to reduce a decrease in accuracy of detection of a grip by a hand of the driver.

Although FIG. 1A to FIG. 11 exemplify one reference electrode 14, the present disclosure is not limited to this example. For example, as shown in FIG. 20 and FIG. 21, reference electrode 14 may be disposed on each of the right side and the left side of rim 20. In this case, two reference electrodes 14 may be electrically connected to heart rate detection circuit 41. FIG. 20 is a block diagram showing steering wheel detection device 3i when reference electrodes 14 are disposed on the right side and the left side of rim 20. FIG. 21 is a diagram showing rim 20 in which each of the electrodes shown in FIG. 20 is disposed, and cross sections of rim 20. (a) of FIG. 21 shows rim 20 in which each of the electrodes shown in FIG. 20 is disposed and that is in the neutral position. (b) of FIG. 21 shows a cross section of rim 20 taken along line B10-B10 shown in (a) of FIG. 21. (c) of FIG. 21 shows a cross section of rim 20 taken along line A10-A10 shown in (a) of FIG. 21.

As shown in FIG. 12 to FIG. 19, in steering wheel detection devices 3e to 3h each including grip detection electrode 15 and grip detection circuit 43, reference electrode 14 may also be disposed on each of the right side and the left side of rim 20. In this case, two reference electrodes 14 may be electrically connected to heart rate detection circuit 41.

Reference electrode 14 on the right side of rim 20 may be disposed closer to the inner circumferential side or the outer circumferential side of rim 20 than first sweat detection electrode 12 and first heart rate and sweat detection electrode 13. Reference electrode 14 on the left side of rim 20 may be disposed closer to the inner circumferential side or the outer circumferential side of rim 20 than first heart rate detection electrode 11.

In such a configuration, when a hand of the driver grips reference electrode 14 and the right side or the left side of rim 20 simultaneously, it is expected to obtain an accurate heart rate signal or sweat signal.

Although FIG. 1A to FIG. 21 show the examples in which steering wheel detection devices 3 to 3i each include first heart rate and sweat detection electrode 13, the present disclosure is not limited to these examples.

For example, first heart rate and sweat detection electrode 13 may be caused not to detect both a heart rate and sweating, and electrodes may be configured to detect a heart rate and sweating, respectively. In this case, a heart rate detection electrode and a sweat detection electrode may be disposed close to each other in the portion in which first heart rate and sweat detection electrode 13 is disposed.

### Advantageous Effects

Next, the advantageous effects of steering wheel detection devices 3 to 3i according to the present embodiment are described.

As stated above, the driver's heart rate signal measurement device according to PTL 1 can be improved upon. Since it is possible to detect only the heart rate of the driver, there has been a demand for further detecting the sweating state of the driver other than the heart rate of the driver. In view of this, steering wheel detection devices 3 to 3i according to technique 1 of the present embodiment each include first heart rate detection electrode 11, first sweat detection electrode 12, first heart rate and sweat detection electrode 13, and reference electrode 14 that are disposed in rim 20 of steering wheel 2 provided in vehicle 1. In a plan view of rim 20 in a neutral position: first heart rate detection electrode 11 is disposed on one of a right side or a left side of rim 20; first heart rate and sweat detection electrode 13 is disposed on an other of the left side or the right side of rim 20; and first sweat detection electrode 12 is disposed adjacent to and not in contact with first heart rate and sweat detection electrode 13. In the plan view of rim 20 in the neutral position, reference electrode 14 is disposed on at least one of the right side or the left side of rim 20, reference electrode 14 not being in contact with first heart rate detection electrode 11, first sweat detection electrode 12, or first heart rate and sweat detection electrode 13.

For example, since first heart rate detection electrode 11 and first heart rate and sweat detection electrode 13 obtain weak electrical signals from a body, heart rate signals that are electrical signals are considered susceptible to factors such as a variation in potential inside the body and the influence of environmental noise.

However, according to the present embodiment, since it is possible to expect that heart rate signals for which an influence affected by such factors is reduced are obtained using reference electrode 14 that makes it possible to supply a reference potential, it is possible to detect a change in heart rate of the driver.

Moreover, since first sweat detection electrode 12 is disposed adjacent to first heart rate and sweat detection electrode 13, when the driver grips steering wheel 2, a hand of the driver comes into contact with both first sweat detection electrode 12 and first heart rate and sweat detection electrode 13. For example, it is possible to obtain a sweat signal via the hand of the driver and first heart rate and sweat detection electrode 13 by applying a weak alternating voltage to first sweat detection electrode 12.

Furthermore, since it is possible to expect that sweat signals for which noise included in the sweat signals is reduced are obtained using reference electrode 14, it is possible to detect the sweating state of the hand of the driver.

Accordingly, steering wheel detection devices 3 to 3i make it possible to detect the heart rate and the sweating state of the driver.

Steering wheel detection device 3a according to technique 2 of the present embodiment is steering wheel detection device 3a according to technique 1. In this case, in rim 20, reference electrode 14 is disposed on a same side as first sweat detection electrode 12 and first heart rate and sweat detection electrode 13, and is disposed closer to an inner circumference or an outer circumference of rim 20 than first sweat detection electrode 12 and first heart rate and sweat detection electrode 13 are.

For example, when the driver grips steering wheel 2 with only one hand, it is considered that the driver is likely to grip, with only the right hand, the right side of rim 20 that is a side on which a direction indicator is disposed in right-hand drive vehicle 1, and that the driver is likely to grip, with only the left hand, the left side of rim 20 that is a side on which a direction indicator is disposed in left-hand drive vehicle 1. For this reason, when first sweat detection electrode 12, first heart rate and sweat detection electrode 13, and reference electrode 14 are collectively disposed on the right side of rim 20 in right-hand drive vehicle 1, and first sweat detection electrode 12, first heart rate and sweat detection electrode 13, and reference electrode 14 are collectively disposed on the left side of rim 20 in left-hand drive vehicle 1, even though the driver grips steering wheel 2 with only one hand, it is possible to expect that the sweating state of the driver is detected.

Steering wheel detection devices 3 to 3i according to technique 3 of the present embodiment are steering wheel detection devices 3 to 3i according to technique 1 or 2. In this case, a surface area of reference electrode 14 is larger than a surface area of first heart rate detection electrode 11, a surface area of first sweat detection electrode 12, and a surface area of first heart rate and sweat detection electrode 13.

Accordingly, when the driver grips steering wheel 2 with a hand, the hand of the driver easily comes into contact with not only first heart rate detection electrode 11, first sweat detection electrode 12, and first heart rate and sweat detection electrode 13 but also reference electrode 14 that is larger in area than the areas of these electrodes. For this reason, it is possible to expect that noise derived from the body is reduced, and a highly accurate heart rate and a highly accurate sweating state are measured.

Steering wheel detection devices 3 to 3i according to technique 4 of the present embodiment are steering wheel detection devices 3 to 3i according to any one of techniques 1 to 3. In this case, in the plan view of rim 20, first heart rate detection electrode 11, first sweat detection electrode 12, first heart rate and sweat detection electrode 13, and reference electrode 14 are disposed in a front portion of rim 20 in vehicle 1, or in the plan view of rim 20, at least one of first heart rate detection electrode 11, first sweat detection electrode 12, first heart rate and sweat detection electrode 13, or reference electrode 14 is disposed in an outer circumferential portion of rim 20.

Accordingly, it is possible to dispose reference electrode 14, first heart rate detection electrode 11, first sweat detection electrode 12, and first heart rate and sweat detection electrode 13 at positions that make it easy to be in contact with the palms occupying a large part of the hands of the driver when the hands grip steering wheel 2. For this reason, it is possible to expect that the heart rate and the sweating state are detected more reliably when the hands of the driver grip steering wheel 2.

Steering wheel detection devices 3b and 3f according to technique 5 of the present embodiment are steering wheel detection devices 3b and 3f according to any one of techniques 1 to 4. In this case, steering wheel detection devices 3b and 3f each further include second heart rate detection electrodes 11b1 and 11b2 disposed in spokes 30 of steering wheel 2.

Accordingly, it is possible to dispose first heart rate detection electrode 11 and second heart rate detection electrodes 11b1 and 11b2 in order that, when the hands of the driver grip steering wheel 2, the fingers of the hands come into contact with first heart rate detection electrode 11 and second heart rate detection electrodes 11b1 and 11b2. For this reason, it is possible to expect that the heart rate of the driver is detected stably.

Steering wheel detection device 3c according to technique 6 of the present embodiment is steering wheel detection device 3c according to any one of techniques 1 to 4. In this case, steering wheel detection device 3c further includes second sweat detection electrodes 12b1 and 12b2 disposed in spokes 30 of steering wheel 2.

Accordingly, it is possible to dispose first sweat detection electrode 12 and second sweat detection electrodes 12b1 and 12b2 in order that, when the hands of the driver grip steering wheel 2, the fingers of the hands come into contact with first sweat detection electrode 12 and second sweat detection electrodes 12b1 and 12b2. For this reason, it is possible to expect that the sweating state of the driver is detected stably.

Steering wheel detection devices 3d and 3g according to technique 7 of the present embodiment are steering wheel detection devices 3d and 3g according to any one of techniques 1 to 4. In this case, steering wheel detection devices 3d and 3g each further include: second heart rate and sweat detection electrode 13b that is disposed in spoke 30 of steering wheel 2; second sweat detection electrodes 12b1 and 12b2 that are disposed in spokes 30 of steering wheel 2; sweat detection circuit 42 that detects a sweating state of a driver; heart rate detection circuit 41 that detects a heart rate of the driver; first switch 43a that switches between electrically connecting first sweat detection electrode 12 to sweat detection circuit 42 and electrically connecting second sweat detection electrode 12b1 to sweat detection circuit 42; second switch 43b that switches between electrically connecting first heart rate and sweat detection electrode 13 to sweat detection circuit 42 and heart rate detection circuit 41 and electrically connecting second heart rate and sweat detection electrode 13b to sweat detection circuit 42 and heart rate detection circuit 41; and switch controller 44 that causes first switch 43a and second switch 43b to switch in a coordinated manner between (i) electrically connecting first heart rate and sweat detection electrode 13 to sweat detection circuit 42 and heart rate detection circuit 41 while electrically connecting first sweat detection electrode 12 to sweat detection circuit 42 and (ii) electrically connecting second heart rate and sweat detection electrode 13b to sweat detection circuit 42 and heart rate detection circuit 41 while electrically connecting second sweat detection electrode 12b1 to sweat detection circuit 42.

Accordingly, it is possible to cause first switch 43a and second switch 43b to switch in a coordinated manner between, for example, obtaining a sweat signal from first sweat detection electrode 12 and first heart rate and sweat detection electrode 13 disposed in rim 20 and obtaining a sweat signal from second sweat detection electrode 12b1 and second heart rate and sweat detection electrode 13b disposed in spoke 30. As a result of this, switching makes it possible to obtain a superior sweat signal to detect a sweating state. For this reason, it is possible to expect that the sweating state of the driver is detected stably.

Steering wheel detection devices 3 to 3i according to technique 8 of the present embodiment are steering wheel detection devices 3 to 3i according to any one of techniques 1 to 7. In this case, steering wheel detection devices 3 to 3i each further include: sweat detection circuit 42 that is electrically connected to first sweat detection electrode 12 and first heart rate and sweat detection electrode 13; and heart rate detection circuit 41 that is electrically connected to first heart rate detection electrode 11 and first heart rate and sweat detection electrode 13. Reference electrode 14 is connected to heart rate detection circuit 41 or sweat detection circuit 42. Heart rate detection circuit 41 and sweat detection circuit 42 detect a heart rate and a sweating state simultaneously or by time division.

Accordingly, when a filter circuit is used, it is possible to detect a heart rate and a sweating state simultaneously; or when a filter circuit is not used, it is possible to detect a heart rate and a sweating state by making a heart rate detection timing and a sweating state detection timing different by time division. When the filter circuit is used, whether to detect a heart rate and a sweating state simultaneously or by time division may be selected appropriately according to whether both data on the heart rate and data on the sweating state are always necessary or whether it is desired to detect both data on the heart rate and data on the sweating state sufficiently separately.

Steering wheel detection devices 3e to 3h according to technique 9 of the present embodiment are steering wheel detection devices 3e to 3h according to any one of techniques 1 to 8 each of which include grip detection electrode 15. Grip detection electrode 15 is disposed in rim 20, grip detection electrode 15 not being in contact with first heart rate detection electrode 11, first sweat detection electrode 12, first heart rate and sweat detection electrode 13, or reference electrode 14.

Accordingly, it is possible to detect a grip of steering wheel 2 by a hand of the driver.

Steering wheel detection devices 3 to 3i according to technique 10 of the present embodiment are steering wheel detection devices 3 to 3i according to technique 9. In this case, an alternating voltage supplied to grip detection electrode 15 and an alternating voltage supplied to first sweat detection electrode 12 differ in frequency.

Accordingly, it is possible to detect a grip, a heart rate, and a sweating state simultaneously.

Steering wheel detection devices 3 to 3i according to technique 11 of the present embodiment are steering wheel detection devices 3 to 3i according to any one of techniques 1 to 10. In this case, steering wheel detection devices 3 to 3i each include: heart rate detection circuit 41 that is electrically connected to first heart rate detection electrode 11, first heart rate and sweat detection electrode 13, and reference electrode 14; and sweat detection circuit 42 that is electrically connected to first sweat detection electrode 12 and first heart rate and sweat detection electrode 13.

Accordingly, it is possible to detect the heart rate and the sweating state of the driver.

Steering wheel detection devices 3e to 3h according to technique 12 of the present embodiment are steering wheel detection devices 3e to 3h according to technique 9 or 10. In this case, steering wheel detection devices 3e to 3h each include: heart rate detection circuit 41 that is electrically connected to first heart rate detection electrode 11, first heart rate and sweat detection electrode 13, and reference electrode 14; sweat detection circuit 42 that is electrically connected to first sweat detection electrode 12 and first heart rate and sweat detection electrode 13; and grip detection circuit 43 that is electrically connected to grip detection electrode 15.

Accordingly, it is possible to detect the heart rate and the sweating state of the driver and a grip of steering wheel 2 by a hand of the driver.

While the embodiment has been described herein above, it is to be appreciated that various changes in form and detail may be made without departing from the spirit and scope of the present disclosure as presently or hereafter claimed.

### Other Variations

Although the steering wheel detection device according to the present disclosure has been described above based on the embodiment, the present disclosure is not limited to the embodiment. Forms obtained by various modifications to the embodiment that can be conceived by a person skilled in the art may be included in the scope of the present disclosure, as long as they do not depart from the essence of the present disclosure.

For example, although the case in which each of the aforementioned electrodes is disposed on the front side of the rim in the aforementioned steering wheel detection device is exemplified, the present disclosure is not limited to this case. Each electrode may be disposed closer to the inner circumference or the outer circumference of the rim. Each electrode may be disposed on the back side of the rim.

Forms obtained by various modifications to the embodiment that can be conceived by a person skilled in the art as well as forms realized by combining the constituent elements and the functions in the embodiment without departing from the essence of the present disclosure are included in the present disclosure.

### Further Information About Technical Background to This Application

The disclosure of the following patent application including specification, drawings, and claims is incorporated herein by reference in their entirety: Japanese Patent Application No. 2024-198781 filed on Nov. 14, 2024.

### Industrial Applicability

The present disclosure is useful for a steering wheel provided in a vehicle.

1. A steering wheel detection device comprising:
a first heart rate detection electrode, a first sweat detection electrode, a first heart rate and sweat detection electrode, and a reference electrode that are disposed in a rim of a steering wheel provided in a vehicle,
wherein in a plan view of the rim in a neutral position:
the first heart rate detection electrode is disposed on one of a right side or a left side of the rim;
the first heart rate and sweat detection electrode is disposed on an other of the left side or the right side of the rim; and
the first sweat detection electrode is disposed adjacent to and not in contact with the first heart rate and sweat detection electrode, and
in the plan view of the rim in the neutral position, the reference electrode is disposed on at least one of the right side or the left side of the rim, the reference electrode not being in contact with the first heart rate detection electrode, the first sweat detection electrode, or the first heart rate and sweat detection electrode.

2. The steering wheel detection device according to claim 1,
wherein in the rim, the reference electrode is disposed on a same side as the first sweat detection electrode and the first heart rate and sweat detection electrode, and is disposed closer to an inner circumference or an outer circumference of the rim than the first sweat detection electrode and the first heart rate and sweat detection electrode are.

3. The steering wheel detection device according to claim 1,
wherein a surface area of the reference electrode is larger than a surface area of the first heart rate detection electrode, a surface area of the first sweat detection electrode, and a surface area of the first heart rate and sweat detection electrode.

4. The steering wheel detection device according to claim 1,
wherein in the plan view of the rim, the first heart rate detection electrode, the first sweat detection electrode, the first heart rate and sweat detection electrode, and the reference electrode are disposed in a front portion of the rim in the vehicle, or in the plan view of the rim, at least one of the first heart rate detection electrode, the first sweat detection electrode, the first heart rate and sweat detection electrode, or the reference electrode is disposed in an outer circumferential portion of the rim.

5. The steering wheel detection device according to claim 1, further comprising:
a second heart rate detection electrode that is disposed in a spoke of the steering wheel.

6. The steering wheel detection device according to claim 1, further comprising:
a second sweat detection electrode that is disposed in a spoke of the steering wheel.

7. The steering wheel detection device according to claim 1, further comprising:
a second heart rate and sweat detection electrode that is disposed in a spoke of the steering wheel;
a second sweat detection electrode that is disposed in a spoke of the steering wheel;
a sweat detection circuit that detects a sweating state of a driver;
a heart rate detection circuit that detects a heart rate of the driver;
a first switch that switches between electrically connecting the first sweat detection electrode to the sweat detection circuit and electrically connecting the second sweat detection electrode to the sweat detection circuit;
a second switch that switches between electrically connecting the first heart rate and sweat detection electrode to the sweat detection circuit and the heart rate detection circuit and electrically connecting the second heart rate and sweat detection electrode to the sweat detection circuit and the heart rate detection circuit; and
a switch controller that causes the first switch and the second switch to switch in a coordinated manner between (i) electrically connecting the first heart rate and sweat detection electrode to the sweat detection circuit and the heart rate detection circuit while electrically connecting the first sweat detection electrode to the sweat detection circuit and (ii) electrically connecting the second heart rate and sweat detection electrode to the sweat detection circuit and the heart rate detection circuit while electrically connecting the second sweat detection electrode to the sweat detection circuit.

8. The steering wheel detection device according to claim 1, further comprising:
a sweat detection circuit that is electrically connected to the first sweat detection electrode and the first heart rate and sweat detection electrode; and
a heart rate detection circuit that is electrically connected to the first heart rate detection electrode and the first heart rate and sweat detection electrode,
wherein the reference electrode is connected to the heart rate detection circuit or the sweat detection circuit, and
the heart rate detection circuit and the sweat detection circuit detect a heart rate and a sweating state simultaneously or by time division.

9. The steering wheel detection device according to claim 1, further comprising:
a grip detection electrode,
wherein the grip detection electrode is disposed in the rim, the grip detection electrode not being in contact with the first heart rate detection electrode, the first sweat detection electrode, the first heart rate and sweat detection electrode, or the reference electrode.

10. The steering wheel detection device according to claim 9,
wherein an alternating voltage supplied to the grip detection electrode and an alternating voltage supplied to the first sweat detection electrode differ in frequency.

11. The steering wheel detection device according to claim 1, comprising:
a heart rate detection circuit that is electrically connected to the first heart rate detection electrode, the first heart rate and sweat detection electrode, and the reference electrode; and
a sweat detection circuit that is electrically connected to the first sweat detection electrode and the first heart rate and sweat detection electrode.

12. The steering wheel detection device according to claim 9, comprising:
a heart rate detection circuit that is electrically connected to the first heart rate detection electrode, the first heart rate and sweat detection electrode, and the reference electrode;
a sweat detection circuit that is electrically connected to the first sweat detection electrode and the first heart rate and sweat detection electrode; and
a grip detection circuit that is electrically connected to the grip detection electrode.
